SEQUENCE LISTING (77 sequences)

SEQ ID NO: 1 - protein, 20 aa, synthetic construct
  source 1..20: note = amino acid sequence of GSK3-binding ligand
MVEPQKFAEELIHRLEAVQR

SEQ ID NO: 2 - protein, 15 aa, synthetic construct
  source 1..15: note = amino acid sequence of GSK3-binding ligand
MEELIDRSLLEAVRR

SEQ ID NO: 3 - protein, 15 aa, synthetic construct
  source 1..15: note = amino acid sequence of linker
AAVLEYLTAEILELA

SEQ ID NO: 4 - protein, 15 aa, synthetic construct
  source 1..15: note = amino acid sequence fuseed with C-terminal
KLAAALEHHHHHHHH

SEQ ID NO: 5 - DNA, 1289 bp, synthetic construct
  source 1..1289: mol_type = other DNA; note = CDS of hGSK3beta
aggagatatacatatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccaccaagcttgcggccgcac

SEQ ID NO: 6 - DNA, 33 bp, synthetic construct
  source 1..33: mol_type = other DNA; note = upstream primer for hGSK3beta expression vector
aggagatatacatatgtcagggcggcccagaac

SEQ ID NO: 7 - DNA, 35 bp, synthetic construct
  source 1..35: mol_type = other DNA; note = downstream primer for hGSK3beta expression vector
gtgcggccgcaagcttggtggagttggaagctgat

SEQ ID NO: 8 - DNA, 34 bp, synthetic construct
  source 1..34: mol_type = other DNA; note = upstream primer for N-terminal domain-knockout hGSK3beta expression vector
aggagatatacatatgagtgcaaagcagctggtc

SEQ ID NO: 9 - DNA, 35 bp, synthetic construct; identical to SEQ ID NO: 7
  source 1..35: mol_type = other DNA; note = upstream primer for intermediate domain-knockout hGSK3beta expression vector

SEQ ID NO: 10 - DNA, 36 bp, synthetic construct
  source 1..36: mol_type = other DNA; note = upstream fusion primer for intermediate domain-knockout hGSK3beta expression vector
ctctgtgactttggagcacatccttggactaaggtc

SEQ ID NO: 11 - DNA, 36 bp, synthetic construct
  source 1..36: mol_type = other DNA; note = downstream fusion primer for intermediate domain-knockout hGSK3beta expression vector
gaccttagtccaaggatgtgctccaaagtcacagag

SEQ ID NO: 12 - DNA, 18 bp, synthetic construct
  source 1..18: mol_type = other DNA; note = pET41b backbone upstream fusion primer
ttccgaccccgaactcca

SEQ ID NO: 13 - DNA, 24 bp, synthetic construct
  source 1..24: mol_type = other DNA; note = pET41b backbone downstream fusion primer
ttttaatacagcagtatcaggatc

SEQ ID NO: 14 - DNA, 33 bp, synthetic construct; identical to SEQ ID NO: 6
  source 1..33: mol_type = other DNA; note = upstream primer for N-terminal domain hGSK3beta expression vector

SEQ ID NO: 15 - DNA, 38 bp, synthetic construct
  source 1..38: mol_type = other DNA; note = downstream primer for N-terminal domain hGSK3beta expression vector
gtgcggccgcaagctttccaaagtcacagagttttaat

SEQ ID NO: 16 - DNA, 34 bp, synthetic construct; identical to SEQ ID NO: 8
  source 1..34: mol_type = other DNA; note = upstream primer for intermediate domain hGSK3beta expression vector

SEQ ID NO: 17 - DNA, 39 bp, synthetic construct
  source 1..39: mol_type = other DNA; note = downstream primer for intermediate domain hGSK3beta expression vector
gtgcggccgcaagcttcttaatttgagggaatttaaatt

SEQ ID NO: 18 - DNA, 33 bp, synthetic construct; identical to SEQ ID NO: 6
  source 1..33: mol_type = other DNA; note = upstream primer for C-terminal domain hGSK3beta expression vector

SEQ ID NO: 19 - DNA, 35 bp, synthetic construct
  source 1..35: mol_type = other DNA; note = downstream primer for C-terminal domain hGSK3beta expression vector
aggagatatacatatggcacatccttggactaagg

SEQ ID NO: 20 - DNA, 35 bp, synthetic construct
  source 1..35: mol_type = other DNA; note = upstream primer for AtBIN2 expression vector
aggagatatacatatggctgatgataaggagatgc

SEQ ID NO: 21 - DNA, 37 bp, synthetic construct
  source 1..37: mol_type = other DNA; note = downstream primer for AtBIN2 expression vector
gtgcggccgcaagcttagttccagattgattcaagaa

SEQ ID NO: 22 - DNA, 35 bp, synthetic construct; identical to SEQ ID NO: 20
  source 1..35: mol_type = other DNA; note = upstream primer for N-terminal domain AtBIN2 expression vector

SEQ ID NO: 23 - DNA, 36 bp, synthetic construct
  source 1..36: mol_type = other DNA; note = downstream primer for N-terminal domain AtBIN2 expression vector
gtgcggccgcaagcttgccaaagtcacagattttga

SEQ ID NO: 24 - DNA, 36 bp, synthetic construct
  source 1..36: mol_type = other DNA; note = upstream primer for intermediate domain AtBIN2 expression vector
aggagatatacatatgagtgcgaaacagctcgttaa

SEQ ID NO: 25 - DNA, 37 bp, synthetic construct
  source 1..37: mol_type = other DNA; note = downstream primer for intermediate domain AtBIN2 expression vector
gtgcggccgcaagcttctttatctgtggaaacctgaa

SEQ ID NO: 26 - DNA, 35 bp, synthetic construct
  source 1..35: mol_type = other DNA; note = upstream primer for C-terminal domain AtBIN2 expression vector
aggagatatacatatggcacatccctggcacaaga

SEQ ID NO: 27 - DNA, 37 bp, synthetic construct; identical to SEQ ID NO: 21
  source 1..37: mol_type = other DNA; note = downstream primer for C-terminal domain AtBIN2 expression vector

SEQ ID NO: 28 - DNA, 660 bp, synthetic construct
  source 1..660: mol_type = other DNA; note = codon-optimized CDS sequence of Human PD-L1
ttcacggttacggtaccgaaagatctgtacgttgtcgaatacggttccaacatgaccattgaatgtaaattcccggtagaaaaacagctggacctggcggcactgattgtttactgggaaatggaagacaaaaatatcatccagttcgtacacggtgaagaagacctgaaagttcagcacagctcttaccgtcagcgtgctcgtctgctgaaggaccagctgtccctgggcaacgctgccctgcagatcactgatgtaaaactgcaggatgcgggcgtctaccgttgtatgatcagctacggcggcgcggactataaacgcattactgtcaaagtcaacgcgccgtacaacaaaatcaaccagcgcattctggtggtggacccggttacctctgaacacgaactgacctgtcaagctgagggctacccgaaagccgaagtcatctggacgtcctctgaccaccaggtgctgtccggtaaaacgacgactaccaactccaaacgtgaggaaaagctgttcaacgttacctctaccctgcgcattaacactaccactaacgaaatcttctactgcaccttccgtcgtctggacccggaagaaaaccacactgcggaactggtcatcccggagctgccgctggcacacccgccgaacgaacgt

SEQ ID NO: 29 - DNA, 126 bp, synthetic construct
  source 1..126: mol_type = other DNA; note = codon-optimized CDS sequence of Abeta42
gatgcagaattccgtcatgactccggttacgaagtgcatcaccaaaaactggttttcttcgcagaggatgttggcagcaacaaaggtgcaatcatcggtctgatggttggtggtgtggttatcgca

SEQ ID NO: 30 - DNA, 669 bp, synthetic construct
  source 1..669: mol_type = other DNA; note = codon-optimized CDS sequence of novel coronavirus spike protein XS
cgtgtgcagccgaccgaatctatcgttcgttttccgaacattaccaacctgtgtccatttcacgaagtgttcaacgcaacgacctttgcgtctgtatacgcgtggaaccgtaaacgtatctctaattgtgtggccgactatagcgttatctacaacttcgcaccgttcttcgcattcaaatgttacggtgttagcccgaccaaactgaacgacctgtgctttaccaacgtttatgctgacagcttcgttatccgtggtaacgaagtgtctcagatcgcgccgggccaaaccggtaacatcgctgactacaactacaaactgccggacgacttcaccggctgcgtgattgcatggaactctaacaaactggattctaaacctagcggtaactacaactatctgtaccgcctgttccgcaaatctaaactgaaaccgtttgaacgcgacatcagcactgaaatctaccaggccggtaacaaaccgtgcaacggtgtagcgggctccaattgttacagcccgctgcagtcttacggtttccgccctacctacggtgtgggccatcagccgtaccgtgtagtcgtactgtctttcgaactgctgcatgcgccggcgaccgtctgtggcccgaaaaaatctactaatctggttaaaaacaagtgcgtgaacttt

SEQ ID NO: 31 - DNA, 38 bp, synthetic construct
  source 1..38: mol_type = other DNA; note = upstream primer for htPD-L1 expression vector
ggatggttcaactagtatgttcacggttacggtaccga

SEQ ID NO: 32 - DNA, 36 bp, synthetic construct
  source 1..36: mol_type = other DNA; note = downstream primer for htPD-L1 expression vector
ctagttattgctcagcttaacgttcgttcggcgggt

SEQ ID NO: 33 - DNA, 39 bp, synthetic construct
  source 1..39: mol_type = other DNA; note = upstream primer for hAbeta42 expression vector
ggatggttcaactagtatggatgcagaattccgtcatga

SEQ ID NO: 34 - DNA, 38 bp, synthetic construct
  source 1..38: mol_type = other DNA; note = downstream primer for hAbeta42 expression vector
ctagttattgctcagcttatgcgataaccacaccacca

SEQ ID NO: 35 - DNA, 35 bp, synthetic construct
  source 1..35: mol_type = other DNA; note = upstream primer for XS expression vector
ggatggttcaactagtatgcgtgtgcagccgaccg

SEQ ID NO: 36 - DNA, 41 bp, synthetic construct
  source 1..41: mol_type = other DNA; note = downstream primer for XS expression vector
ctagttattgctcagcttaaaagttcacgcacttgttttta

SEQ ID NO: 37 - protein, 69 aa, synthetic construct
  source 1..69: note = amino acid sequence of novel coronavirus spike glycoprotein ligand
STIEEQAKTFLDKFNHEAEDLFYQSSLASWNYNTNITEENVQNMNNAGDKWSAFLKEQSTLAQMYPLQE

SEQ ID NO: 38 - protein, 74 aa, synthetic construct
  source 1..74: note = amino acid sequence of hPD-L1 ligand
HVVWHRESPSGQTDTLAAFPEDRSQPGQDARFRVTQLPNGRDFHMSVVRARRNDSGTYVCGVISLAPKIQIKES

SEQ ID NO: 39 - protein, 81 aa, synthetic construct
  source 1..81: note = amino acid sequence of hPD-L1 ligand
GFTFSSYIMMWVRQAPGKGLEWVSSIYPSGGITFYADTVKGRFTISRDNSKNTLYLQMNSLRAEDTAVYYCARIKLGTVTT

SEQ ID NO: 40 - protein, 72 aa, synthetic construct
  source 1..72: note = amino acid sequence of hPD-L1 ligand
SDSWIHWVRQAPGKGLEWVAWISPYGGSTYYADSVKGRFTISADTSKNTAYLQMNSLRAEDTAVYYCARRHW

SEQ ID NO: 41 - protein, 33 aa, synthetic construct
  source 1..33: note = amino acid sequence of hAbeta42 ligand
AMSWVRQAPGKGLEWVSAINASGTRTYYADSVK

SEQ ID NO: 42 - protein, 76 aa, synthetic construct
  source 1..76: note = amino acid sequence of hAbeta42 ligand
GFTFSRYSMSWVRQAPGKGLELVAQINSVGSSTYYPDTVKGRFTISRDNAKNTLYLQMNSLRAEDTAVYYCASGDY

SEQ ID NO: 43 - protein, 72 aa, synthetic construct
  source 1..72: note = amino acid sequence of hAbeta42 ligand
WIEWIKQRPGHGLEWIGEVLPGSGKSNHNANFKGRATFTADTASNTAYMQLSSLTSEDSAVYYCAREGSNNN

SEQ ID NO: 44 - protein, 100 aa, synthetic construct
  source 1..100: note = amino acid sequence of XS ligand
GRSLRLSCAASGFTFDDYAMHWVRQAPGKGLEWVSGVSWNSGTIGYADSVKGRFTISRDNAKNSLYLHMRSLRAEDTALYYCAKAVEMVRGLMGLGADPE

SEQ ID NO: 45 - protein, 64 aa, synthetic construct
  source 1..64: note = amino acid sequence of XS ligand
SSAVAWYQQKPGKAPKLLIYSASDLYSGVPSRFSGSRSGTDFTLTISSLQPEDFATYYCQQSYR

SEQ ID NO: 46 - DNA, 297 bp, synthetic construct
  source 1..297: mol_type = other DNA; note = DNA sequence of BLC
atggaggaattaatagatagaagtcttctagaggcggttcgtagagctgctgttctagaatatttgacagctgaaattcttgagttggctagtactatcgaggagcaagccaaaacatttcttgataagtttaaccacgaggctgaggaccttttttatcaatcctctttggcctcctggaattataatactaacatcaccgaggagaatgttcaaaatatgaacaacgctggggataagtggtctgcatttctcaaggagcaatctacacttgcacagatgtacccactgcaggaa

SEQ ID NO: 47 - DNA, 327 bp, synthetic construct
  source 1..327: mol_type = other DNA; note = DNA sequence of GLP
atggttgagccgcagaaattcgccgaagaactgatccaccgtctggaagctgttcagcgtgcggccgtgctggaatacctgactgcagaaatcctggaactggctcatgtagtttggcaccgcgaatctccgtccggtcagaccgataccctggctgcatttccggaagaccgctctcagccgggtcaggatgctcgtttccgcgttacccagctgcctaacggccgtgatttccacatgagcgtcgtgcgtgcccgccgtaacgattccggtacctacgtatgcggcgttatttctctggcgccgaaaatccagatcaaagaaagc

SEQ ID NO: 48 - DNA, 348 bp, synthetic construct
  source 1..348: mol_type = other DNA; note = DNA sequence of GLPH
atggttgagccgcagaaattcgccgaagaactgatccaccgtctggaagctgttcagcgtgcggccgtgctggaatacctgactgcagaaatcctggaactggctggttttactttcagcagctacattatgatgtgggtacgtcaggcaccgggtaaaggcctggagtgggtaagcagcatctatccgagcggtggtatcaccttctacgctgacaccgtcaaaggtcgtttcaccatctctcgcgacaacagcaagaacaccctgtacctgcagatgaactccctgcgtgcggaggatactgcagtctactattgcgcccgtatcaaactgggtacggtcactacc

SEQ ID NO: 49 - DNA, 321 bp, synthetic construct
  source 1..321: mol_type = other DNA; note = DNA sequence of GLPL
atggttgagccgcagaaattcgccgaagaactgatccaccgtctggaagctgttcagcgtgcggccgtgctggaatacctgactgcagaaatcctggaactggctagcgattcttggattcactgggtacgtcaggctccgggcaaaggtctggaatgggttgcatggatttctccgtacggcggttccacgtactatgctgactccgtgaaaggtcgcttcacgatctccgcagacacctccaaaaacaccgcctacctgcagatgaattctctgcgcgcggaagacaccgcagtatactactgcgctcgtcgccactgg

SEQ ID NO: 50 - DNA, 204 bp, synthetic construct
  source 1..204: mol_type = other DNA; note = DNA sequence of GLAm
atggttgagccgcagaaattcgccgaagaactgatccaccgtctggaagctgttcagcgtgcggccgtgctggaatacctgactgcagaaatcctggaactggctgcgatgtcttgggttcgccaagcaccaggcaaaggcctggaatgggtgagcgcgatcaacgcttctggtacccgcacctactacgcggactccgttaaa

SEQ ID NO: 51 - DNA, 333 bp, synthetic construct
  source 1..333: mol_type = other DNA; note = DNA sequence of GLAmH
atggttgagccgcagaaattcgccgaagaactgatccaccgtctggaagctgttcagcgtgcggccgtgctggaatacctgactgcagaaatcctggaactggctggtttcactttctcccgctactctatgtcctgggttcgtcaggctccgggtaaaggcctggaactggttgcgcagatcaactccgttggtagctctacttactatccggacacggttaagggtcgtttcaccatctcccgtgataacgcgaaaaacacgctgtacctgcagatgaattctctgcgtgctgaagatactgcggtttactattgtgcatccggtgactac

SEQ ID NO: 52 - DNA, 321 bp, synthetic construct
  source 1..321: mol_type = other DNA; note = DNA sequence of GLAmM
atggttgagccgcagaaattcgccgaagaactgatccaccgtctggaagctgttcagcgtgcggccgtgctggaatacctgactgcagaaatcctggaactggcttggatcgaatggattaaacagcgcccaggtcatggtctggaatggatcggtgaagtactgccgggttctggtaagtctaaccacaacgcgaacttcaaaggccgtgccactttcactgcagatactgcttccaacacggcgtatatgcagctgtcttccctgacctccgaagattctgcggtttactactgcgctcgtgaaggctctaacaacaac

SEQ ID NO: 53 - DNA, 312 bp, synthetic construct
  source 1..312: mol_type = other DNA; note = DNA sequence of GLC
atggttgagccgcagaaattcgccgaagaactgatccaccgtctggaagctgttcagcgtgcggccgtgctggaatacctgactgcagaaatcctggaactggctagtactatcgaggagcaagccaaaacatttcttgataagtttaaccacgaggctgaggaccttttttatcaatcctctttggcctcctggaattataatactaacatcaccgaggagaatgttcaaaatatgaacaacgctggggataagtggtctgcatttctcaaggagcaatctacacttgcacagatgtacccactgcaggaa

SEQ ID NO: 54 - DNA, 405 bp, synthetic construct
  source 1..405: mol_type = other DNA; note = DNA sequence of GLCH
atggttgagccgcagaaattcgccgaagaactgatccaccgtctggaagctgttcagcgtgcggccgtgctggaatacctgactgcagaaatcctggaactggctggtcgttctctgcgtctgagctgtgccgcatctggtttcacttttgatgattacgctatgcattgggtgcgtcaagcgccaggtaaaggtctggaatgggtttctggcgtgagctggaactctggtaccatcggctatgcggattccgtaaagggtcgtttcactatcagccgcgataacgcgaagaactccctgtacctgcacatgcgttccctgcgtgcagaagacaccgcgctgtactactgcgccaaagcagtagaaatggttcgcggcctgatgggtctgggtgcagatccggaa

SEQ ID NO: 55 - DNA, 297 bp, synthetic construct
  source 1..297: mol_type = other DNA; note = DNA sequence of GLCM
atggttgagccgcagaaattcgccgaagaactgatccaccgtctggaagctgttcagcgtgcggccgtgctggaatacctgactgcagaaatcctggaactggcttcctctgcagtagcatggtaccaacagaaaccgggtaaagcgccgaaactgctgatttactctgcgtctgatctgtactccggcgttccttcccgttttagcggttctcgttctggcaccgacttcaccctgaccatctcttctctgcagccagaagacttcgcgacttactactgccagcaatcctaccgt

SEQ ID NO: 56 - DNA, 38 bp, synthetic construct
  source 1..38: mol_type = other DNA; note = upstream primer for BLC expression vector
aggagatatacatatggaggaattaatagatagaagtc

SEQ ID NO: 57 - DNA, 35 bp, synthetic construct
  source 1..35: mol_type = other DNA; note = downstream primer for BLC expression vector
gtgcggccgcaagcttttcctgcagtgggtacatc

SEQ ID NO: 58 - DNA, 35 bp, synthetic construct
  source 1..35: mol_type = other DNA; note = upstream primer for GLP expression vector
aggagatatacatatggtagaaccgcagaaattcg

SEQ ID NO: 59 - DNA, 37 bp, synthetic construct
  source 1..37: mol_type = other DNA; note = downstream primer for GLP expression vector
gtgcggccgcaagcttgctttctttgatctggatttt

SEQ ID NO: 60 - DNA, 35 bp, synthetic construct
  source 1..35: mol_type = other DNA; note = upstream primer for GLPH expression vector
atcctggaactggctggttttactttcagcagcta

SEQ ID NO: 61 - DNA, 33 bp, synthetic construct
  source 1..33: mol_type = other DNA; note = downstream primer for GLPH expression vector
tgcggccgcaagcttggtagtgaccgtacccag

SEQ ID NO: 62 - DNA, 16 bp, synthetic construct
  source 1..16: mol_type = other DNA; note = pET41b-GL-His backbone upstream primer
aagcttgcggccgcac

SEQ ID NO: 63 - DNA, 19 bp, synthetic construct
  source 1..19: mol_type = other DNA; note = pET41b-GL-His backbone downstream primer
agccagttccaggatttct

SEQ ID NO: 64 - DNA, 35 bp, synthetic construct
  source 1..35: mol_type = other DNA; note = upstream primer for GLPL expression vector
atcctggaactggctagcgattcttggattcactg

SEQ ID NO: 65 - DNA, 31 bp, synthetic construct
  source 1..31: mol_type = other DNA; note = downstream primer for GLPL expression vector
tgcggccgcaagcttccagtggcgacgagcg

SEQ ID NO: 66 - DNA, 71 bp, synthetic construct
  source 1..71: mol_type = other DNA; note = upstream primer for GLAm expression vector
atcctggaactggctgcgatgtcttgggttcgccaagcaccaggcaaaggcctggaatgggtgagcgcgat

SEQ ID NO: 67 - DNA, 73 bp, synthetic construct
  source 1..73: mol_type = other DNA; note = downstream primer for GLAm expression vector
tgcggccgcaagctttttaacggagtccgcgtagtaggtgcgggtaccagaagcgttgatcgcgctcacccat

SEQ ID NO: 68 - DNA, 34 bp, synthetic construct
  source 1..34: mol_type = other DNA; note = upstream primer for GLAmH expression vector
atcctggaactggctggtttcactttctcccgct

SEQ ID NO: 69 - DNA, 34 bp, synthetic construct
  source 1..34: mol_type = other DNA; note = downstream primer for GLAmH expression vector
tgcggccgcaagcttgtagtcaccggatgcacaa

SEQ ID NO: 70 - DNA, 36 bp, synthetic construct
  source 1..36: mol_type = other DNA; note = upstream primer for GLAmM expression vector
atcctggaactggcttggatcgaatggattaaacag

SEQ ID NO: 71 - DNA, 35 bp, synthetic construct
  source 1..35: mol_type = other DNA; note = downstream primer for GLAmM expression vector
tgcggccgcaagcttgttgttgttagagccttcac

SEQ ID NO: 72 - DNA, 35 bp, synthetic construct
  source 1..35: mol_type = other DNA; note = upstream primer for GLC expression vector
aggagatatacatatggttgaaccgcagaaattcg

SEQ ID NO: 73 - DNA, 35 bp, synthetic construct
  source 1..35: mol_type = other DNA; note = downstream primer for GLC expression vector
gtgcggccgcaagcttttcctgcagcgggtacatt

SEQ ID NO: 74 - DNA, 33 bp, synthetic construct
  source 1..33: mol_type = other DNA; note = upstream primer for GLCH expression vector
atcctggaactggctggtcgttctctgcgtctg

SEQ ID NO: 75 - DNA, 33 bp, synthetic construct
  source 1..33: mol_type = other DNA; note = downstream primer for GLCH expression vector
tgcggccgcaagcttttccggatctgcacccag

SEQ ID NO: 76 - DNA, 34 bp, synthetic construct
  source 1..34: mol_type = other DNA; note = upstream primer for GLCM expression vector
atcctggaactggcttcctctgcagtagcatggt

SEQ ID NO: 77 - DNA, 33 bp, synthetic construct
  source 1..33: mol_type = other DNA; note = downstream primer for GLCM expression vector
tgcggccgcaagcttacggtaggattgctggca

(19) United States
(12) Patent Application Publication
(10) Pub. No.: US 2026/0049292 A1
(43) Pub. Date: Feb. 19, 2026

(54) USE OF GLYCOGEN SYNTHASE KINASE-3 (GSK3) AS PROTEASE, AND GSK3-BASED PROTEOLYSIS-TARGETING CHIMERA (PROTAC) AND PREPARATION METHOD AND USE THEREOF

(71) Applicant: Peng GUO, Haidian District, Beijing (CN)

(72) Inventor: Peng GUO, Haidian District, Beijing (CN)

(21) Appl. No.: 18/881,654

(22) PCT Filed: Aug. 13, 2024
(86) PCT No.: PCT/CN2024/111590

§ 371 Date: Jan. 6, 2025

(51) Int. Cl. C12N 9/12

(52) U.S. Cl. CPC C12N 9/12; C12Y 207/11026

(57) ABSTRACT

Use of glycogen synthase kinase-3 (GSK3) as a protease, and a GSK3-based proteolysis-targeting chimera (PROTAC) and a preparation method and use thereof are provided, belonging to the technical field of proteolysis. The GSK3 and an N-terminal domain, an intermediate domain, or a C-terminal domain thereof have a protease activity and can achieve efficient proteolysis. Based on the GSK3 and other similar proteases, PROTACs can be developed to achieve efficient targeted proteolysis of a target protein.

### CROSS-REFERENCE TO RELATED APPLICATION

This patent application is a national stage application of International Patent Application No: PCT/CN2024/111590 filed on Aug. 13, 2024, the disclosure of which is incorporated by reference herein in its entirety as part of the present application.

### REFERENCE TO SEQUENCE LISTING

A computer readable XML file entitled “GWPCTP20240806321-sequence listing”, which was created on Dec. 19, 2024, with a file size of about 91,324 bytes, contains the sequence listing for this application, has been filed with this application, and is hereby incorporated by reference in its entirety.

### TECHNICAL FIELD

The present disclosure belongs to the technical field of proteolysis, and specifically relates to the use of glycogen synthase kinase-3 (GSK3) as a protease, and a GSK3-based proteolysis-targeting chimera (PROTAC) and a preparation method and use thereof.

### BACKGROUND

Glycogen synthase kinase-3 (GSK3) is a serine/threonine kinase ubiquitous in eukaryotes. With over 100 substrates, GSK3 is also known as the busiest kinase in most cells. GSK3 was first discovered in rabbit skeletal muscle in 1980. As its name suggests, the GSK3 inactivates glycogen synthase by phosphorylation. In the following 40 years, the important role of GSK3 was found in many signal transduction pathways of animals and plants. However, reports on the function of GSK3 so far are limited to kinase properties, and there are no reports on other enzyme properties.

### SUMMARY

A purpose of the present disclosure is to provide use of GSK3 as a protease, and a GSK3-based proteolysis-targeting chimera (PROTAC) and a preparation method and use thereof. In the present disclosure, the GSK3 is used as a protease to achieve proteolysis of proteins.

The present disclosure provides the use of GSK3 as a protease.

The present disclosure further provides use of GSK3 as a protease having any one or two or more functions of aspartate protease, serine protease, threonine protease, cysteine protease, and metalloprotease.

The present disclosure provides use of an N-terminal domain, an intermediate domain, or a C-terminal domain of GSK3 as a protease.

The present disclosure provides use of an N-terminal domain of GSK3 as a protease having any one or two or more functions of aspartate protease, serine protease, threonine protease, cysteine protease, and metalloprotease.

The present disclosure provides use of an intermediate domain of GSK3 as a protease having any one or two or more functions of aspartate protease, serine protease, threonine protease, and cysteine protease.

The present disclosure provides use of a C-terminal domain of GSK3 as a protease having any one or two or more functions of aspartate protease, serine protease, and cysteine protease.

The present disclosure provides a protease-based PROTAC, where the protease-based PROTAC is formed by ligating two ligands with a linker, and the two ligands include a target protein-binding ligand and a protease-binding ligand.

The present disclosure provides a GSK3-based PROTAC, where the GSK3-based PROTAC is formed by ligating two ligands with a linker, and the two ligands include a target protein-binding ligand and a GSK3-binding ligand.

Preferably, when the GSK3-binding ligand is a polypeptide and GSK3 is a human protein, the GSK3-binding ligand includes the amino acid sequence shown in SEQ ID NO: 1.

Preferably, when the GSK3-binding ligand is a polypeptide and GSK3 is an Arabidopsis thaliana protein, the GSK3-binding ligand includes the amino acid sequence shown in SEQ ID NO: 2.

Preferably, the linker includes the amino acid sequence shown in SEQ ID NO: 3.

Preferably, when the target protein-binding ligand is a polypeptide, the target protein-binding ligand includes an amino acid sequence encoding a protein that interacts with a target protein on a binding surface.

Preferably, C-terminal of the amino acid sequence encoding the protein that interacts with the target protein on the binding surface is further fused with the amino acid sequence shown in SEQ ID NO: 4.

The present disclosure provides use of the protease-based PROTAC or the GSK3-based PROTAC in preparation of a targeting agent for proteolysis.

The present disclosure provides a method for proteolysis of a target protein based on the protease-based PROTAC or the GSK3-based PROTAC, including the following steps: mixing the protease-based PROTAC or the GSK3-based PROTAC with a target protein-containing sample to allow targeted proteolysis of the target protein.

Preferably, the targeted proteolysis is conducted at 36° C. to 37° C. when GSK3 is a human protein.

Preferably, the targeted proteolysis is conducted at 26° C. to 28° C. when GSK3 is an Arabidopsis thaliana protein.

Preferably, cisplatin is preferably added into a resulting mixed system.

The present disclosure provides use of cisplatin in preparation of a reagent for enhancing a protease activity of GSK3.

The present disclosure provides use of cisplatin in preparation of a reagent for enhancing a proteolytic effect of GSK3 on a target protein mediated by the GSK3-based PROTAC.

The present disclosure provides use of GSK3 as a protease. In the present disclosure, the GSK3 is used as a protease to achieve proteolysis of proteins. Further, the GSK3 and an N-terminal domain, an intermediate domain, or a C-terminal domain thereof can achieve efficient proteolysis. Based on the GSK3, PROTACs can be developed to achieve efficient targeted proteolysis of a target protein.

### BRIEF DESCRIPTION OF THE DRAWINGS

To describe the technical solutions in examples of the present disclosure or in the prior art more clearly, the accompanying drawings required in the examples are briefly described below: Apparently, the accompanying drawings in the following description show merely some examples of the present disclosure, and other drawings can be derived from these accompanying drawings by those of ordinary skill in the art without creative efforts.

FIG. 1 shows the immunoblotting results of human and Arabidopsis thaliana GSK3 and domain-deleted variants thereof expressed and purified by Escherichia coli provided in the present disclosure;

FIG. 2 shows the immunoblotting results of human PD-L1, human Aβ42, and novel coronavirus spike glycoprotein XS expressed and purified by E. coli provided in the present disclosure:

FIG. 3 shows the immunoblotting results of GPTAC expressed and purified by E. coli provided in the present disclosure:

FIG. 4 shows a protease activity analysis of human and Arabidopsis thaliana GSK3 and domain-deleted variants thereof expressed and purified by E. coli provided in the present disclosure:

FIG. 5A-FIG. 5E show the factors affecting protease activity of human-derived GSK3 provided by the present disclosure: where FIG. 5A is the analysis of inhibitors of the protease activity of human GSK3: FIG. 5B is the analysis of activators of the protease activity of human GSK3: FIG. 5C is the analysis of the protease properties of the N-terminal domain of human GSK3: FIG. 5D is the analysis of the protease properties of the intermediate domain of human GSK3: FIG. 5E is the analysis of the protease properties of the C-terminal domain of human GSK3:

FIG. 6A-FIG. 6D show that GPTAC provided in the present disclosure guides the GSK3 in proteolysis of target protein: where FIG. 6A is a GPTAC specific to the novel coronavirus spike glycoprotein NS-mFc, which guides Arabidopsis thaliana GSK3 in proteolysis of NS-mFc: FIG. 6B is a GPTAC with different affinities to PD-L1, which guides human GSK3 in proteolysis of PD-L1; FIG. 6C is a GPTAC with different affinities to Aβ42, which guides human GSK3 in proteolysis of Aβ42: FIG. 6D is a GPTAC with different affinities to the novel coronavirus spike glycoprotein XS, which guides human GSK3 in proteolysis of XS; and

FIG. 7A-FIG. 7C show that cisplatin-promoted GPTAC provided in the present disclosure guides human GSK3 in proteolysis of target protein: where FIG. 7A is cisplatin-promoted PD-L1-specific GPTAC to guide human GSK3 in proteolysis of PD-L1; FIG. 7B is cisplatin-promoted Aβ42-specific GPTAC to guide human GSK3 in proteolysis of Aβ42; FIG. 7C is cisplatin-promoted novel coronavirus spike glycoprotein XS-specific GPTAC to guide human GSK3 in proteolysis of XS.

### DETAILED DESCRIPTION OF THE EMBODIMENTS

The present disclosure provides use of GSK3 as a protease. In the present disclosure, the GSK3 preferably includes human GSK3 (human glycogen synthase kinase 3-β subtype, hGSK3β) and Arabidopsis thaliana GSK3 (AtBIN2). It is found that GSK3 is a multifunctional enzyme that also has protease activity and possesses three independent protease domains. GSK3 at least has the function of an endopeptidase and can achieve proteolysis of proteins as a protease.

The present disclosure further provides use of GSK3 as a protease having any one or two or more functions of aspartate protease, serine protease, threonine protease, cysteine protease, and metalloprotease.

The present disclosure provides use of an N-terminal domain, an intermediate domain, or a C-terminal domain of GSK3 as a protease. The N-terminal and intermediate domains of GSK3 are extremely active, while the C-terminal domain thereof is relatively weak. Therefore, the GSK3 has an overall higher degradation efficiency compared to that of all other proteases.

The present disclosure provides use of an N-terminal domain of GSK3 as a protease having any one or two or more functions of aspartate protease, serine protease, threonine protease, cysteine protease, and metalloprotease.

The present disclosure provides use of an intermediate domain of GSK3 as a protease having any one or two or more functions of aspartate protease, serine protease, threonine protease, and cysteine protease.

The present disclosure provides use of a C-terminal domain of GSK3 as a protease having any one or two or more functions of aspartate protease, serine protease, and cysteine protease.

The present disclosure provides a protease-based PROTAC, where the protease-based PROTAC is formed by ligating two ligands with a linker, and the two ligands include a target protein-binding ligand and a protease-binding ligand. In the present disclosure, the protease preferably includes GSK3. The protease may also be other proteases having protease activity similar to that of GSK3. The protease-based PROTAC has physical properties similar to those of PROTAC and can be a polypeptide or a PROTAC-like organic compound. The protease-based PROTAC must be soluble in water and can be synthesized and purified in vitro.

The present disclosure provides a GSK3-based PROTAC (as known as GPTAC), where the GSK3-based PROTAC is formed by ligating two ligands with a linker, and the two ligands include a target protein-binding ligand and a GSK3-binding ligand. That is, the GPTAC includes three parts, namely a GSK3 ligand, a linker, and a target protein ligand. The GPTAC has physical properties similar to those of PROTAC and can be a polypeptide or a PROTAC-like organic compound. Most of the interacting proteins of proteases are their substrates, making it difficult to target the target protein. The GPTAC, which is similar to the effect of PROTAC on E3 ubiquitin ligase and target protein, can combine GSK3 and target protein together and directly utilize the protease activity of GSK3 to achieve efficient and specific proteolysis of target protein.

In the present disclosure, when the GSK3-binding ligand is a polypeptide and GSK3 is a human protein, the GSK3-binding ligand includes preferably an amino acid sequence shown in SEQ ID NO: 1 (MVEPQKFAEELIHRLEAVQR). When the GSK3-binding ligand is a polypeptide and GSK3 is an Arabidopsis thaliana protein, the GSK3-binding ligand includes preferably an amino acid sequence shown in SEQ ID NO: 2 (MEELIDRSLLEAVRR). It is discovered that GSK3 has a region that can bind to Axin but does not degrade Axin. The N-terminal domain of GPTAC is preferably derived from an Axin domain that interacts with GSK3. The binding ability of GSK3 and Axin can achieve the binding of PROTAC and GSK3, thereby achieving the proteolysis of target protein.

In the present disclosure, the linker includes preferably an amino acid sequence shown in SEQ ID NO: 3 (AAVLEYLTAEILELA). The linker domain is derived from the IBIVU database (www.ibi.vu.nl/programs/linkerdbwww/).

In the present disclosure, when the target protein-binding ligand is a polypeptide, the target protein-binding ligand includes preferably an amino acid sequence encoding a protein that interacts with a target protein on a binding surface. C-terminal of the amino acid sequence encoding the protein that interacts with the target protein on the binding surface is further fused with the amino acid sequence shown in SEQ ID NO: 4 (KLAAALEHHHHHHHH). That is, the target protein ligand is the amino acid sequence of the protein (including antibodies) on the binding surface that interacts with the target protein in an existing crystal structure, and KLAAALEHHHHHHHH (SEQ ID NO: 4) is fused to the C-terminal of the ligand.

In the present disclosure, the GPTAC must be soluble in water and can be synthesized and purified in vitro. There is no particular limitation on the in vitro synthesis and purification method. Polypeptide-type GPTAC can be purified using various protein expression systems, such as E. coli, yeast, insects, and HEK293 cell lines. Organic GPTAC is preferably synthesized by referring to a synthesis method of PROTAC.

In the present disclosure, the protease-based PROTAC is not limited to GPTAC, and the GPTAC is not limited to the specific polypeptide-type GPTAC listed above. Other derivative types of GPTAC obtained based on the same construction principle all fall within the protection scope of the present disclosure. For example, the N-terminal of the polypeptide-type GPTAC can be replaced with a sequence similar to the existing Axin sequence; or the N-terminal of the polypeptide-type GPTAC can be replaced with other sequences, such that GPTAC can target other proteases. Alternatively, the linker of the polypeptide-type GPTAC can be replaced with other sequences. Alternatively, the protease-based PROTAC may be an organic GPTAC, or another type, such as a nucleic acid peptide.

The present disclosure provides use of the GSK3-based PROTAC in preparation of a targeting agent for proteolysis. Proteases are generally specific, some of which are substrate specific, but more of which are sequence site specific. This means that most of the interacting proteins of protease are its substrates, making it difficult to target the target protein. The present disclosure reveals that GSK3 has protease activity, and the GPTAC (GSK3 PROTAC) derived from GSK3, which has a similar effect of PROTAC on E3 ubiquitin ligase and target protein, can effectively combine GSK3 and target protein together to achieve efficient and specific proteolysis of target protein. GPTAC, like PROTAC, can be directly, quickly, and effectively applied in various eukaryotic organisms such as animals and plants. Moreover, GSK3 has three independent protease domains, and the N-terminal and intermediate domains show extremely strong protease activity. Meanwhile, GSK3 is almost globally distributed at the cellular level and almost constitutively expressed at the tissue level. As a result, GPTAC has great potential to directly utilize all PROTACs through the efficient and specific degradation ability for target proteins and wider expression range. The application areas of GPTAC preferably include: (a) all areas covered by existing PROTAC; (b) proteolysis of all proteins in the blood (harmful proteins in the blood, such as those from free bacteria, cancer cells, secretions of the body itself, and viruses); (c) suicide of harmful cells in the body (such as cancer cells, senescent cells, apoptotic cells, pyroptotic cells, and necrotic cells); (d) repair and self-rescue of cells attacked by viruses or other damaged and senescent cells; and (e) other areas.

When the GPTAC is applied to animals, polypeptide-type GPTAC is preferably administered by injection, and organic GPTAC can be administered by injection or orally. When the GPTAC is applied to plants, polypeptide-type GPTAC is preferably applied in a transgenic manner, and organic GPTAC can be applied via vascular injection, topically, or through the roots.

PROTAC relies on the natural proteolysis system in the cell (ubiquitin-proteasome system) to achieve specific proteolysis of target proteins. A few protein-targeted proteolysis systems utilize lysosomes, such as AUTAC. Since PROTACs are highly dependent on the ubiquitin-proteasome system, redundancy of E3 ubiquitin ligases can lead to off-target effects. The function of GPTAC is based on the protease activity of GSK3. First, GSK3 is widely distributed in both animals and plants and is almost distributed throughout the cell (cell membrane, cytoplasm, nucleus, mitochondria, and chloroplasts). In human beings, hGSK3B is constitutively distributed (except muscle and soft tissues); in Arabidopsis thaliana, AtBIN2 (a homolog of GSK3) is distributed throughout the life cycle and in most tissues (except pollen). Secondly, whether in animals or plants, the homologous proteins of GSK3 have extremely high amino acid homology. Since GPTAC directly utilizes the protease activity of GSK3, it is likely that homologous proteins of GSK3 may further increase the efficiency of GPTAC. The only difference in design between GPTAC and PROTAC is that the ligand of the E3 ubiquitin ligase in PROTAC is replaced with the GSK3 ligand. Therefore, it is only necessary to optimize the GSK3 ligand into the best organic small molecule to replace all PROTACs with corresponding GPTACs at an extremely low R&D cost, thereby improving the efficiency and range of target protein proteolysis while retaining all the advantages of PROTAC. After entering the cell, GPTAC can directly guide GSK3 to degrade the target protein; while PROTAC may first guide E3 ubiquitin ligase to ubiquitinate the target protein, and then the proteasome can recognize and degrade the target protein. Therefore, the reaction steps after GPTAC enters the cell can be much fewer than those of PROTAC. Experiments in vitro show that once GPTAC is added to the reaction system, the reaction ends instantly and the target protein can be degraded immediately. However, organic GPTACs cannot be degraded and can be recycled like PROTACs. Moreover, since GPTACs with different affinities for the target protein may behave similarly, it is impossible to distinguish their differences on a time scale (once GPTAC is added to the reaction system, the corresponding target protein is instantly degraded. Just a few seconds is not enough to determine which GPTAC is more effective in directing GSK3 to degrade target proteins. In other words, the GPTAC in the present example has a difference in affinity with the target protein, which does not affect a proteolysis efficiency of the target protein by GSK3). In conclusion, GPTAC works extremely efficiently in vitro.

PROTAC can only degrade proteins within cells (including membrane proteins) and cannot eliminate endogenous and exogenous proteins in the blood. The GSK3B that GPTAC depends on is present in human platelets, peripheral blood mononuclear cells, and serum. This means that GPTAC has great potential to degrade any protein in the blood. Harmful proteins in the blood may come from free bacteria, cancer cells, the body's own secretions, and viruses. The in vitro experiments in the examples of the present disclosure can achieve efficient proteolysis of PD-L1 protein on the surface of cancer cells, Alzheimer's disease-causing peptide Aβ42, and novel coronavirus spike glycoprotein.

GPTAC has all the advantages of PROTAC over gene editing:

Gene editing changes the information in the genome. At this stage, it is impossible to fully analyze the function of any gene or protein. There is a high probability that there are knowledge blind spots and risks after application. GPTAC is an instant application technology that can efficiently target and degrade proteins during use. Once use is terminated, the targeted proteolysis effect may gradually disappear. Not only does the technology not alter the genome, it can also be adjusted at any time and quickly reapplied.

Gene editing may have certain off-target effects. GPTAC has high specificity and affinity for the target protein, greatly reducing off-target effects.

Gene editing has limited ability to knock out multiple genes (1. multiple unrelated genes; 2. multiple homologous genes or gene family genes) simultaneously. On one hand, there are limitations on the vector capacity and compatibility of multiple vectors for co-transformation; on the other hand, for a single sequence, to achieve one-time knockout of a gene family or multiple homologous genes, it is necessary to find and rely on the sequence consistency of key sites, which is highly difficult. However, GPTAC has no limitation on the number of target proteins that can be degraded simultaneously. On one hand, GPTAC is based on the recognition of the spatial structure of proteins, such that a reasonable design (high specificity or high affinity) can simultaneously degrade the proteins corresponding to the gene family or multiple homologous proteins or isoproteins. On the other hand, as mentioned above, GPTAC has an extremely high proteolysis efficiency in vitro and is currently in the form of a consumable polypeptide (i.e., it will eventually be degraded by GSK3 to leave only the N-terminal GSK3 ligand). Therefore, there is no limitation on the number of GPTACs that can be used, that is, more GPTAC units targeting different proteins can be used at the same time. Even if GPTAC is optimized and upgraded into a non-degradable small molecule in the future, its efficient proteolysis of target proteins and the optimization of the usage ratio of different units may give it a huge advantage in terms of usage quantity.

In animals, there are still many limitations to gene editing. For example, relevant norms and ethical principles need to be strictly observed. GPTAC does not change the genome and therefore does not pose any ethical risks. GPTAC is time-sensitive, which means it takes effect when used and becomes invalid when discontinued. In addition, at this stage, the technology is of the consumable type (polypeptide type), and almost all components (except linker) can be replaced with corresponding animal sources, with extremely low immunogenicity. After optimization and upgrading, GPTAC can draw on the advantages of all related PROTACs to minimize immunogenicity. The optimization and upgrading refers to designing GPTAC into an organic small molecule. Furthermore, for existing PROTACs, the corresponding GPTACs can be obtained by partially replacing the E3 ubiquitin ligase ligand with the GSK3 organic ligand. Because the optimized and upgraded GPTAC is highly similar to the PROTAC structure it borrowed, the optimized GPTAC can have similar immunogenicity as PROTAC.

In plants, there are also still many limitations to gene editing. First, it is difficult to establish callus or somatic embryo systems of different species, and the efficiency of gene editing is limited. Second, there is great variability in the effects and effectiveness of different gene editing techniques. At present, GPTAC is still in the original polypeptide form with a molecular weight of about 8 kDa to 16 kDa, while the GPTAC in the examples contains 80 to 160 amino acids, corresponding to 240 nt to 480 nt of DNA. Due to the limited examples, GPTAC can at least be used in plants through transgenic overexpression; compared with gene editing, under the same vector load limit, GPTAC transgene has more independent sequence units; under the same number of sequence units, GPTAC can target more target proteins. Secondly, plants have a vascular system similar to animal blood vessels, but they also have intercellular filaments that animal cells do not have, which can achieve efficient transport of substances between cells. Since the design compositions of GPTAC and PROTAC are highly similar, the current molecular weight of PROTAC is only 700 Da to 1,200 Da, and the molecular weight of molecular glue is less than 500 Da (for example, ARV-110 from Arvinas for the treatment of prostate cancer is the world's first PROTAC drug to enter clinical trials and has a molecular weight of 812 Da. ARV-471 from Arvinas for the treatment of breast cancer has a molecular weight of 724 Da. Currently, there are only three molecular glue drugs (thalidomide, lenalidomide, and pomalidomide) approved for the treatment of solid tumors and hematological malignancies in the world, with molecular weights of 258 Da, 259 Da, and 273 Da, respectively), such that the molecular weight of the iteratively updated GPTAC has the potential to be less than 1 kDa. Although the plasmodesmata have a size exclusion limit (SEL) of 800 Da to 1,000 Da, the optimized GPTAC may have higher probability of passing through the plasmodesmata and the vascular system. In addition, the cell permeability of small molecules is extremely high, and can achieve more efficient targeted protein proteolysis than the current GPTAC.

The present disclosure provides a method for proteolysis of a target protein based on the GSK3-based PROTAC, including the following steps: mixing the GSK3-based PROTAC with a target protein-containing sample to allow targeted proteolysis of the target protein.

In the present disclosure, the targeted proteolysis is conducted at preferably 36° C. to 37° C., more preferably 37° C. when GSK3 is a human protein.

In the present disclosure, the targeted proteolysis is conducted at preferably 26° C. to 28° C., more preferably 28° C. when GSK3 Desirable is an Arabidopsis thaliana protein.

In the present disclosure, cisplatin is preferably added into a resulting mixed system.

The present disclosure provides use of cisplatin in preparation of a reagent for enhancing a protease activity of GSK3.

The present disclosure provides use of cisplatin in preparation of a reagent for enhancing a proteolytic effect of GSK3 on a target protein mediated by the GSK3-based PROTAC.

In order to further illustrate the present disclosure, the use of GSK3 as a protease, and the GSK3-based PROTAC and the preparation method and the use thereof provided by the present disclosure are described in detail below with reference to the accompanying drawings and examples, but the accompanying drawings and the examples should not be construed as limiting the protection scope of the present disclosure.

### Example 1

Gene cloning, protein expression, and purification of hGSK3B, domain-knockout hGSK3β variants, single domain hGSK3β variants, AtBIN2, and single domain AtBIN2 variants
1. Basic information of the hGSK3β (P49841, in UniProt) gene was that it encoded 420 amino acids.
2. Basic information of BIN2 (AT4G18710, in TAIR) gene was that it encoded 380 amino acids.
3. The hGSK3β, domain-knockout hGSK3β variants, single domain hGSK3β variants, AtBIN2, and single domain AtBIN2 variants were subjected to gene cloning.
   (1) Synthesis of hGSK3β CDS

The CDS of hGSK3β was synthesized (codon-optimized without the TGA stop codon) with restriction enzyme site NdeI at the N-terminal and HindIII at the C-terminal for ligation into a pET41b vector to generate a C-terminal His tag. The fragment was commissioned to be synthesized by Beijing Tsingke Biotech Co., Ltd. The sequence is shown in SEQ ID NO: 5:

AGGAGATATACATATGTCAGGGCGGCCCAGAACCACCTCCTTTGCGGAGAGCT
GCAAGCCGGTGCAGCAGCCTTCAGCTTTTGGCAGCATGAAAGTTAGCAGAGACAAG
GACGGCAGCAAGGTGACAACAGTGGTGGCAACTCCTGGGCAGGGTCCAGACAGGCC
ACAAGAAGTCAGCTATACAGACACTAAAGTGATTGGAAATGGATCATTTGGTGTGGTA
TATCAAGCCAAACTTTGTGATTCAGGAGAACTGGTCGCCATCAAGAAAGTATTGCAG
GACAAGAGATTTAAGAATCGAGAGCTCCAGATCATGAGAAAGCTAGATCACTGTAAC
ATAGTCCGATTGCGTTATTTCTTCTACTCCAGTGGTGAGAAGAAAGATGAGGTCTATCT
TAATCTGGTGCTGGACTATGTTCCGGAAACAGTATACAGAGTTGCCAGACACTATAGT
CGAGCCAAACAGACGCTCCCTGTGATTTATGTCAAGTTGTATATGTATCAGCTGTTCCG
AAGTTTAGCCTATATCCATTCCTTTGGAATCTGCCATCGGGATATTAAACCGCAGAACC
TCTTGTTGGATCCTGATACTGCTGTATTAAAACTCTGTGACTTTGGAAGTGCAAAGCA
GCTGGTCCGAGGAGAACCCAATGTTTCGTATATCTGTTCTCGGTACTATAGGGCACCA
GAGTTGATCTTTGGAGCCACTGATTATACCTCTAGTATAGATGTATGGTCTGCTGGCTG
TGTGTTGGCTGAGCTGTTACTAGGACAACCAATATTTCCAGGGGATAGTGGTGTGGAT
CAGTTGGTAGAAATAATCAAGGTCCTGGGAACTCCAACAAGGGAGCAAATCAGAGA
AATGAACCCAAACTACACAGAATTTAAATTCCCTCAAATTAAGGCACATCCTTGGACT
AAGGTCTTCCGACCCCGAACTCCACCGGAGGCAATTGCACTGTGTAGCCGTCTGCTG
GAGTATACACCAACTGCCCGACTAACACCACTGGAAGCTTGTGCACATTCATTTTTTG
ATGAATTACGGGACCCAAATGTCAAACTACCAAATGGGCGAGACACACCTGCACTCT
TCAACTTCACCACTCAAGAACTGTCAAGTAATCCACCTCTGGCTACCATCCTTATTCC
TCCTCATGCTCGGATTCAAGCAGCTGCTTCAACCCCCACAAATGCCACAGCAGCGTC
AGATGCTAATACTGGAGACCGTGGACAGACCAATAATGCTGCTTCTGCATCAGCTTCC
AACTCCACCAAGCTTGCGGCCGCAC.

(2) Extraction of RNA from the Inflorescence Axis of Arabidopsis thaliana and Reverse Transcription into cDNA

When the plant was close to 20 cm in height, the inflorescence axis was cut without flowers. The inflorescence axis was ground with liquid nitrogen, and total RNA was extracted with an RNA extraction kit (Plant RNA Rapid Extraction Kit RN38-EASYspin Plus, Aidlab). The extracted total RNA was reverse transcribed into cDNA using a reverse transcription kit (PC18-TRUEscript 1st Strand cDNA Synthesis Kit, Aidlab).

### (3) Primer Design

Primers with restriction enzyme sites at both ends were designed according to the sequence characteristics and ligated to a pET41b vector to generate a C-terminal His tag. The upstream primer had an NdeI restriction site, and the downstream primer had a HindIII restriction site. All primers were entrusted to Sangon Biotech (Shanghai) Co., Ltd. for synthesis.
(a). Primer design for hGSK3β expression vector (pET41b-GSK3β-His):

upstream primer:
(SEQ ID NO: 6)
AGGAGATATACATATGTCAGGGCGGCCCAGAAC;
and
downstream primer:
(SEQ ID NO: 7)
GTGCGGCCGCAAGCTTGGTGGAGTTGGAAGCTGAT.

(b) Primer design for N-terminal domain-knockout hGSK3β expression vector (pET41b-GSK3β-Δ202-His):

upstream primer:
(SEQ ID NO: 8)
AGGAGATATACATATGAGTGCAAAGCAGCTGGTC;
and
downstream primer:
(SEQ ID NO: 9)
GTGCGGCCGCAAGCTTGGTGGAGTTGGAAGCTGAT.

(c) Primer design for intermediate domain-knockout hGSK3β expression vector (pET41b-GSK3β-Δ95-His):

upstream fusion primer:
(SEQ ID NO: 10)
CTCTGTGACTTTGGAGCACATCCTTGGACTAAGGTC;
and
downstream fusion primer:
(SEQ ID NO: 11)
GACCTTAGTCCAAGGATGTGCTCCAAAGTCACAGAG.
pET41b backbone upstream fusion primer:
(SEQ ID NO: 12)
TTCCGACCCCGAACTCCA;
and
pET41b backbone downstream fusion primer:
(SEQ ID NO: 13)
TTTTAATACAGCAGTATCAGGATC.

(d) Primer design for N-terminal domain hGSK3β expression vector (pET41b-GSK3β-Δ218-His):

upstream primer:
(SEQ ID NO: 14)
AGGAGATATACATATGTCAGGGCGGCCCAGAAC;
and
downstream primer:
(SEQ ID NO: 15)
GTGCGGCCGCAAGCTTTCCAAAGTCACAGAGTTTTAAT.

(e) Primer design for intermediate domain hGSK3β expression vector (pET41b-GSK3β-On95-His):

upstream primer:
(SEQ ID NO: 16)
AGGAGATATACATATGAGTGCAAAGCAGCTGGTC;
and
downstream primer:
(SEQ ID NO: 17)
GTGCGGCCGCAAGCTTCTTAATTTGAGGGAATTTAAATT.

(f) Primer design for C-terminal domain hGSK3β expression vector (pET41b-GSK3β-On123-His):

upstream primer:
(SEQ ID NO: 18)
AGGAGATATACATATGTCAGGGCGGCCCAGAAC;
and
downstream primer:
(SEQ ID NO: 19)
AGGAGATATACATATGGCACATCCTTGGACTAAGG.

(g.). Primer design for AtBIN2 expression vector (pET41b-BIN2-His):

upstream primer:
(SEQ ID NO: 20)
AGGAGATATACATATGGCTGATGATAAGGAGATGC;
and
downstream primer:
(SEQ ID NO: 21)
GTGCGGCCGCAAGCTTAGTTCCAGATTGATTCAAGAA.

(h) Primer design for N-terminal domain AtBIN2 expression vector (pFT41b-BIN2-Δ194-His)

upstream primer:
(SEQ ID NO: 22)
AGGAGATATACATATGGCTGATGATAAGGAGATGC;
and
downstream primer:
(SEQ ID NO: 23)
GTGCGGCCGCAAGCTTGCCAAAGTCACAGATTTTGA.

(i) Primer design for intermediate domain AtBIN2 expression vector (pET41b-BIN2-On95-His):

upstream primer:
(SEQ ID NO: 24)
AGGAGATATACATATGAGTGCGAAACAGCTCGTTAA;
and
downstream primer:
(SEQ ID NO: 25)
GTGCGGCCGCAAGCTTCTTTATCTGTGGAAACCTGAA.

(j) Primer design for C-terminal domain AtBIN2 expression vector (pET41b-BIN2-Δ281-His):

upstream primer:
(SEQ ID NO: 26)
AGGAGATATACATATGGCACATCCCTGGCACAAGA;
and
downstream primer:
(SEQ ID NO: 27)
GTGCGGCCGCAAGCTTAGTTCCAGATTGATTCAAGAA.

### (4) PCR Amplification of the Target Gene

In a 50 μL PrimeSTAR Max (TAKARA, R045B, preparation method referred to reagent instructions) reaction system, hGSK3β and its variant genes were expressed using the synthetic GSK3β CDS as a template, and the vector for the intermediate domain-knockout hGSK3β (pET41b-GSK3β-Δ95-His) was expressed using pET41b-GSK3β-His (construction method: with the synthesized hGSK3β CDS as a template, the CDS of hGSK3β was ligated to a linear vector pET41b that was double-digested with NdeI and HindIII using the upstream and downstream primers of the hGSK3β expression vector (pET41b-GSK3β-His)) as a template. AtBIN2 and its variant genes were expressed using Arabidopsis thaliana cDNA as a template. The primers were used as amplification primers for PCR amplification, and the PCR reaction conditions were: initial denaturation at 98° C. for 4 min; thermal cycle at 98° C. for 30 s, annealing at 55° C. for 15 s, extension at 72° C. for 5 s/kb, 30 cycles; and extension at 72° C. for 10 min.

(5) The target fragment was amplified by PCR. After confirming the DNA amplification product of the target gene by 1% agarose gel electrophoresis, an obtained amplified product was recovered and purified using a PCR product purification kit (DR02-PCR, Aidlab).

### 4. Construction of a Recombinant Plasmid

The vector pET41b (purchased from Thermo Fisher) was double-digested with NdeI and HindIII (purchased from NEB), and the obtained backbone fragment and the DNA fragment purified in step 3 were homologously recombined using a seamless cloning kit (D7010S from Beyotime).

A ligation product was transformed into E. coli DH5α (purchased from Sangon Biotech (Shanghai) Co., Ltd.), spread on solid LB medium containing 30 μg/mL kanamycin and cultured upside down at 37° C. overnight. Single colonies were selected and cultured in liquid LB medium containing 30 μg/mL kanamycin at 37° C., 220 rpm overnight with shaking. The recombinant plasmid in the bacterial solution was extracted using a plasmid extraction kit (PL02, Aidlab). Plasmid PCR was conducted to verify that the recombinant plasmid contained the target fragment. The constructed recombinant plasmids pET41b-GSK3β-His, pET41b-GSK3β-Δ202-His, pET41b-GSK3β-Δ95-His, pET41b-GSK3β-Δ218-His, pET41b-GSK3β-On95-His, pET41b-GSK3β-On123-His, pET41b-BIN2-His, pET41b-BIN2-Δ194-His, pET41b-BIN2-On95-His, and pET41b-BIN2-Δ281-His were entrusted to Beijing Tsingke Biotech Co., Ltd. for sequencing and identification. The detected sequences were compared with the corresponding references and were completely matched. This indicated that the recombinant plasmid was constructed successfully.

### 5. Expression of Recombinant Plasmid in Host Bacteria

The recombinant plasmid was transferred into the host bacterium E. coli BL21 (purchased from Sangon Biotech (Shanghai) Co., Ltd.), and a single colony was selected and cultured in liquid LB medium containing 30 μg/mL kanamycin at 37° C. and 220 rpm overnight. The next day, the culture was inoculated at a ratio of 1:50 into fresh liquid LB medium containing 30 μg/mL kanamycin and cultured until the OD₆₀₀ was 0.4 to 0.6, and an inducer isopropyl-β-D-thiogalactopyranoside (IPTG) was added to a final concentration of 1 mM. The cells were cultured at 28° C. and 220 rpm for 6 h and the cells were collected by centrifugation (8,000 rpm for 10 min).

### 6. Purification of Recombinant Protein

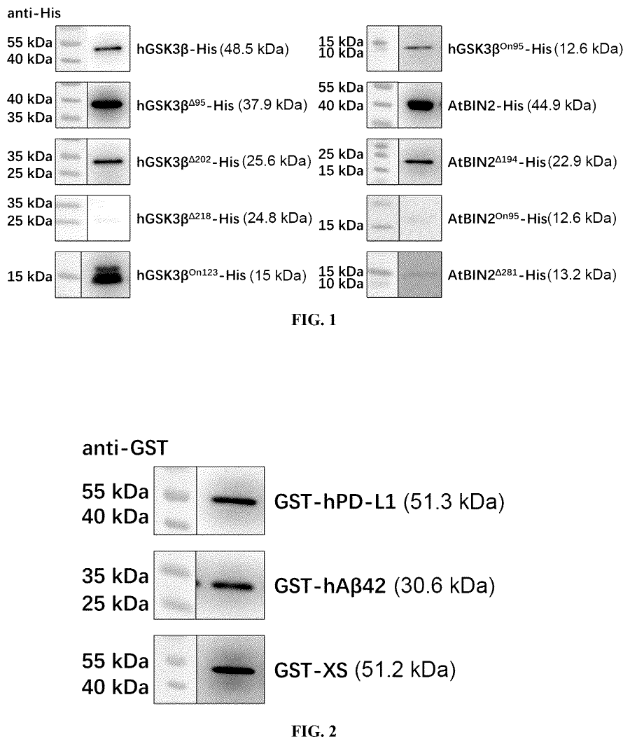

The bacterial cells were resuspended in Ni-NTA lysis buffer (50 mM NaH₂PO₄, 300 mM NaCl, 10 mM imidazol). After ultrasonic disruption (40 apt; ultrasonic treatment for 5 s and stopping for 25 s; totally for 3 min), centrifugation (12,000 rpm, 30 min) was conducted and a supernatant was collected. The Ni-NTA affinity chromatography column (P2233, Beyotime) was equilibrated with 8 column volumes of Ni-NTA lysis buffer; the collected supernatant was then passed through the chromatography column. The chromatography column was washed with 4 column volumes of Ni-NTA lysis buffer; and then washed with 4 column volumes of Ni-NTA washing buffer (50 mM NaH₂PO₄, 300 mM NaCl, 25 mM imidazol). The chromatography column was eluted with 1 column volume of Ni-NTA eluent (50 mM NaH₂PO₄, 300 mM NaCl, 250 mM imidazol). The eluted products were separated by 10% SDS-PAGE and then detected by immunoblotting. The proteins produced by the recombinant plasmid pET41b-GSK3β-His, pET41b-GSK3β-Δ202-His, pET41b-GSK3β-Δ95-His, pET41b-GSK3β-Δ218-His, pET41b-GSK3β-On95-His, pET41b-GSK3β-On123-His, pET41b-BIN2-His, pET41b-BIN2-Δ194-His, pET41b-BIN2-On95-His, and pET41b-BIN2-Δ281-His were named hGSK3β-His, hGSK3β^{Δ202}-His, hGSK3β^{Δ95}-His, hGSK3β^{Δ218}-His, hGSK3β^{On95}-His, hGSK3β^{On123}-His, AtBIN2-His, AtBIN2^{Δ194}-His, AtBIN2^{On95}-His, and AtBIN2^{Δ281}-His, respectively. As shown in FIG. 1, all recombinant proteins were purified correctly.

hGSK3β^{Δ202}-His: hGSK3β lacked the N-terminal domain. If the protein had protease activity, it meant that the intermediate or C-terminal domain of hGSK3β had protease activity; otherwise, it meant that the N-terminal domain of hGSK3β had protease activity.

hGSK3β^{Δ95}-His: hGSK3β lacked the intermediate domain. If the protein had protease activity, it meant that the N-terminal or C-terminal domain of hGSK3β had protease activity; otherwise, it meant that the intermediate domain of hGSK3β had protease activity.

hGSK3β^{Δ218}-His: hGSK3β contained only the N-terminal domain. If the protein had protease activity, it meant that the N-terminal domain of hGSK3β had protease activity; otherwise, it meant that the intermediate or C-terminal domain of hGSK3β had protease activity.

hGSK3β^{On95}-His: hGSK3β contained only the intermediate domain. If the protein had protease activity, it meant that the intermediate domain of hGSK3β had protease activity; otherwise, it meant that the N-terminal or C-terminal domain of hGSK3β had protease activity.

hGSK3β^{On123}-His: hGSK3β contained only the C-terminal domain. If the protein had protease activity, it meant that the C-terminal domain of hGSK3β had protease activity; otherwise, it meant that the N-terminal or intermediate domain of hGSK3β had protease activity.

AtBIN2^{Δ194}-His: AtBIN2 contained only the N-terminal domain. If the protein had protease activity, it meant that the N-terminal domain of AtBIN2 had protease activity; otherwise, it meant that the intermediate or C-terminal domain of AtBIN2 had protease activity.

AtBIN2^{On95}-His: AtBIN2 contained only the intermediate domain. If the protein had protease activity, it meant that the intermediate domain of AtBIN2 had protease activity; otherwise, it meant that the N-terminal or C-terminal domain of AtBIN2 had protease activity.

AtBIN2^{Δ281}-His: AtBIN2 contained only the C-terminal domain. If the protein had protease activity, it meant that the C-terminal domain of AtBIN2 had protease activity; otherwise, it meant that the N-terminal or intermediate domain of AtBIN2 had protease activity.

### Example 2

Gene cloning, protein expression, and purification of human PD-L1, human Aβ42, and novel coronavirus spike protein XS

1. Human PD-L1 (hPD-L1) (Q9NZQ7, in UniProt)

In the example, the htPD-L1 protein (human truncated programmed death-ligand 1) used was a truncated form of hPD-L1. To facilitate expression and purification in the E. coli system, its codon-optimized CDS sequence (without a stop codon) is shown in SEQ ID NO: 28:

TTCACGGTTACGGTACCGAAAGATCTGTACGTTGTCGAATACGGTTCCAACAT
GACCATTGAATGTAAATTCCCGGTAGAAAAACAGCTGGACCTGGCGGCACTGATTGT
TTACTGGGAAATGGAAGACAAAAATATCATCCAGTTCGTACACGGTGAAGAAGACCT
GAAAGTTCAGCACAGCTCTTACCGTCAGCGTGCTCGTCTGCTGAAGGACCAGCTGTC
CCTGGGCAACGCTGCCCTGCAGATCACTGATGTAAAACTGCAGGATGCGGGCGTCTA
CCGTTGTATGATCAGCTACGGCGGCGCGGACTATAAACGCATTACTGTCAAAGTCAAC
GCGCCGTACAACAAAATCAACCAGCGCATTCTGGTGGTGGACCCGGTTACCTCTGAA
CACGAACTGACCTGTCAAGCTGAGGGCTACCCGAAAGCCGAAGTCATCTGGACGTC
CTCTGACCACCAGGTGCTGTCCGGTAAAACGACGACTACCAACTCCAAACGTGAGG
AAAAGCTGTTCAACGTTACCTCTACCCTGCGCATTAACACTACCACTAACGAAATCTT
CTACTGCACCTTCCGTCGTCTGGACCCGGAAGAAAACCACACTGCGGAACTGGTCAT
CCCGGAGCTGCCGCTGGCACACCCGCCGAACGAACGT.

2. Basic information of human Aβ42: Human Aβ42 (human β-amyloid peptide-42, hAβ42) was a part of the intact protein APP (P05067, in UniProt). hAβ42 encoded 42 amino acids. To facilitate expression and purification in the E. coli system, its CDS sequence after codon optimization (without stop codon) is shown in SEQ ID NO: 29:

GATGCAGAATTCCGTCATGACTCCGGTTACGAAGTGCATCACCAAAAAC
TGGTTTTCTTCGCAGAGGATGTTGGCAGCAACAAAGGTGCAATCATCGG
TCTGATGGTTGGTGGTGTGGTTATCGCA.

### 3. Basic Information of Novel Coronavirus Spike Protein XS

SARS-CoV-2 (XBB) Spike RBD (YP_009724390.1, with mutations G339H, R346T, L3681, S371F, S373P, S375F, T376A, D405N, R408S, K417N, N440K, V445P, G446S, N460K, S477N, T478K, E484A, F486S, F490S, Q498R, N501Y, Y505H). XS protein (receptor-binding region of the spike glycoprotein of the new coronavirus (XBB subspecies)) was a part of the SARS-CoV-2 (XBB) Spike RBD (R319-F541). To facilitate expression and purification in the E. coli system, its codon-optimized CDS sequence (without stop codon) is shown in SEQ ID NO: 30:

CGTGTGCAGCCGACCGAATCTATCGTTCGTTTTCCGAACATTACCAACCTGTG
TCCATTTCACGAAGTGTTCAACGCAACGACCTTTGCGTCTGTATACGCGTGGAACCGT
AAACGTATCTCTAATTGTGTGGCCGACTATAGCGTTATCTACAACTTCGCACCGTTCTT
CGCATTCAAATGTTACGGTGTTAGCCCGACCAAACTGAACGACCTGTGCTTTACCAAC
GTTTATGCTGACAGCTTCGTTATCCGTGGTAACGAAGTGTCTCAGATCGCGCCGGGCC
AAACCGGTAACATCGCTGACTACAACTACAAACTGCCGGACGACTTCACCGGCTGCG
TGATTGCATGGAACTCTAACAAACTGGATTCTAAACCTAGCGGTAACTACAACTATCT
GTACCGCCTGTTCCGCAAATCTAAACTGAAACCGTTTGAACGCGACATCAGCACTGA
AATCTACCAGGCCGGTAACAAACCGTGCAACGGTGTAGCGGGCTCCAATTGTTACAG
CCCGCTGCAGTCTTACGGTTTCCGCCCTACCTACGGTGTGGGCCATCAGCCGTACCGT
GTAGTCGTACTGTCTTTCGAACTGCTGCATGCGCCGGCGACCGTCTGTGGCCCGAAA
AAATCTACTAATCTGGTTAAAAACAAGTGCGTGAACTTT.

4. Gene Cloning of htPD-L1, hAβ42, and XS
(1) Synthesis of htPD-L1 (F19-R238) CDS

To synthesize the CDS of htPD-L1 (without the TGA stop codon), Beijing Tsingke Biotech Co., Ltd. was commissioned to synthesize the fragment.

(2) Synthesis of hAβ42 CDS

To synthesize the CDS of hAβ42 (without the TGA stop codon), Beijing Tsingke Biotech Co., Ltd. was commissioned to synthesize the fragment.

### (3) Synthesis of XS CDS

To synthesize the CDS of XS (without the TGA stop codon), Beijing Tsingke Biotech Co., Ltd. was commissioned to synthesize the fragment.

### (4) Primer Design

Primers with restriction enzyme sites at both ends were designed according to the sequence characteristics and ligated to a pET41b vector to generate an N-terminal GST tag. The upstream primer had a SpeI restriction site, and the downstream primer had a BlpI restriction site. All primers were entrusted to Sangon Biotech (Shanghai) Co., Ltd. for synthesis.
(a). Primer design for htPD-L1 expression vector (pET41b-GST-PD-L1):

upstream primer:
(SEQ ID NO: 31)
GGATGGTTCAACTAGTATGTTCACGGTTACGGTACCGA;
and
downstream primer:
(SEQ ID NO: 32)
CTAGTTATTGCTCAGCTTAACGTTCGTTCGGCGGGT.

(b). Primer design for hAβ42 expression vector (ET41b-GST-Aβ42):

upstream primer:
(SEQ ID NO: 33)
GGATGGTTCAACTAGTATGGATGCAGAATTCCGTCATGA;
and
downstream primer:
(SEQ ID NO: 34)
CTAGTTATTGCTCAGCTTATGCGATAACCACACCACCA.

(c). Primer design for XS expression vector (pET41b-GST-XS):

upstream primer:
(SEQ ID NO: 35)
GGATGGTTCAACTAGTATGCGTGTGCAGCCGACCG;
and
downstream primer:
(SEQ ID NO: 36)
CTAGTTATTGCTCAGCTTAAAAGTTCACGCACTTGTTTTTA.

### (5) PCR Amplification of the Target Gene

In a 50 μL PrimeSTAR Max (TAKARA, the preparation method referred to the reagent instructions) reaction system, the synthesized CDS was used as a template, the primers were used as amplification primers for PCR amplification, and the PCR reaction conditions were: initial denaturation at 98° C. for 4 min; thermal cycle at 98° C. for 30 s, annealing at 55° C. for 15 s, extension at 72° C. for 5 s/kb, 30 cycles; and extension at 72° C. for 10 min.

(6) The target fragment was amplified by PCR. After confirming the DNA amplification product of the target gene by 1% agarose gel electrophoresis, an obtained amplified product was recovered and purified using a PCR product purification kit (DR02-PCR, Aidlab).

### 5. Construction of Target Protein Recombinant Plasmid

A construction method was the same as that in Example 1.

The constructed recombinant plasmids pET41b-GST-PD-L1, pET41b-GST-Aβ42, and pET41b-GST-XS were entrusted to Beijing Tsingke Biotech Co., Ltd. for sequencing and identification. The detected sequences were compared with the corresponding references and were completely matched. This indicated that the recombinant plasmid was constructed successfully.

### 6. Expression of Target Protein Recombinant Plasmid in Host Bacteria

A construction method was the same as that in Example 1.

### 7. Purification of Target Protein Recombinant Protein

A construction method was the same as that in Example 1. The proteins produced by the recombinant plasmid pET41b-GST-PD-L1, pET41b-GST-Aβ42, and pET41b-GST-XS were named GST-htPD-L1, GST-hAβ42, and GST-XS, respectively. As shown in FIG. 2, all recombinant proteins were purified correctly.

### Example 3

Gene cloning, protein expression, and purification of polypeptide-type GPTAC
1. The structural components of all GPTACs included: GSK3 (including BIN2) ligand, linker, and target protein ligand. The naming method was G (GSK3)-L (Linker)-Initial Acronym of POI. For example, there was GLP=G (GSK3)-L (Linker)-P (PD-L1). The GPTAC related to BIN2 was named B (BIN2)-L (Linker)-Initial Acronym of POI. For example, there was BLP=B (BIN2)-L (Linker)-P (PD-L1).
2. Basic information of AtBIN2-related polypeptide-type GPTAC
(1) In all BIN2-related polypeptide-type GPTACs, the BIN2 ligand and linker remained unchanged.

The BIN2 ligand had an amino acid sequence:
(SEQ ID NO: 2)
MEELIDRSLLEAVRR.
The linker had an amino acid sequence:
(SEQ ID NO: 3)
AAVLEYLTAEILELA.

(2) GPTAC targeting the novel coronavirus spike glycoprotein: BLC (COVID-19 Spike RBD)

The novel coronavirus spike glycoprotein ligand had an amino acid sequence:

(SEQ ID NO: 37)
STIEEQAKTFLDKFNHEAEDLFYQSSLASWNYNTNITEENVQNMNNAGD
KWSAFLKEQSTLAQMYPLQE.

3. Basic information of hGSK3-related polypeptide-type GPTAC
(1) In all hGSK3-related polypeptide-type GPTACs, the GSK3 ligand and linker remained unchanged.

The GSK3 ligand had an amino acid sequence:
(SEQ ID NO: 1)
MVEPQKFAEELIHRLEAVQR.
The linker had an amino acid sequence:
(SEQ ID NO: 3)
AAVLEYLTAEILELA.

(2) GPTAC targeting hPD-L1: GLP (hPD-L1)

The hPD-L1 ligand had an amino acid sequence:

(SEQ ID NO: 38)
HVVWHRESPSGQTDTLAAFPEDRSQPGQDARFRVTQLPNGRDFHMSVVR
ARRNDSGTYVCGVISLAPKIQIKES.

(3) GPTAC targeting hPD-L1: GLPH (anti-PD-L1 antibody with High affinity)

The hPD-L1 ligand had an amino acid sequence:

(SEQ ID NO: 39)
GFTFSSYIMMWVRQAPGKGLEWVSSIYPSGGITFYADTVKGRFTISRDN
SKNTLYLQMNSLRAEDTAVYYCARIKLGTVTT.

(4) GPTAC targeting hPD-L1: GLPL (anti-PD-L1 antibody with Low affinity)

The hPD-L1 ligand had an amino acid sequence:

(SEQ ID NO: 40)
SDSWIHWVRQAPGKGLEWVAWISPYGGSTYYADSVKGRFTISADTSKNT
AYLQMNSLRAEDTAVYYCARRHW.

(5) GPTAC targeting hAβ42: GLAm (anti-Aβ42 monoclonal antibody)

The hAβ42 ligand had an amino acid sequence:

(SEQ ID NO: 41)
AMSWVRQAPGKGLEWVSAINASGTRTYYADSVK.

(6) GPTAC targeting hAβ42: GLAmH (anti-Aβ42 monoclonal antibody with High affinity)

The hAβ42 ligand had an amino acid sequence:

(SEQ ID NO: 42)
GFTFSRYSMSWVRQAPGKGLELVAQINSVGSSTYYPDTVKGRFTISRDN
AKNTLYLQMNSLRAEDTAVYYCASGDY.

(7) GPTAC targeting hAβ42: GLAmM (anti-Aβ42 monoclonal antibody with Medium affinity)

The hAβ42 ligand had an amino acid sequence:

(SEQ ID NO: 43)
WIEWIKQRPGHGLEWIGEVLPGSGKSNHNANFKGRATFTADTASNTAYM
QLSSLTSEDSAVYYCAREGSNNN.

(8) GPTAC targeting the novel coronavirus spike glycoprotein: GLC (COVID-19 Spike RBD)

The ligand information of the novel coronavirus spike glycoprotein was the same as that in BLC.
(9) GPTAC targeting the novel coronavirus spike glycoprotein: GLCH (anti-COVID-19 Spike RBD monoclonal antibody with High affinity)

The XS ligand had an amino acid sequence:

(SEQ ID NO: 44)
GRSLRLSCAASGFTFDDYAMHWVRQAPGKGLEWVSGVSWNSGTIGYADS
VKGRFTISRDNAKNSLYLHMRSLRAEDTALYYCAKAVEMVRGLMGLGAD
PE.

(10) GPTAC novel coronavirus spike glycoprotein: GLCM (anti-COVID-19 Spike RBD monoclonal antibody with Medium affinity)

The XS ligand had an amino acid sequence:

(SEQ ID NO: 45)
SSAVAWYQQKPGKAPKLLIYSASDLYSGVPSRFSGSRSGTDFTLTISSL
QPEDFATYYCQQSYR.

4. All GPTAC genes were synthesized. To facilitate expression and purification in the E. coli system, their CDS was codon-optimized and did not contain the DNA sequence corresponding to the fusion His tag. The fragments were commissioned to be synthesized by Beijing Tsingke Biotech Co., Ltd.
(1) DNA sequence of BLC:

(SEQ ID NO: 46)
ATGGAGGAATTAATAGATAGAAGTCTTCTAGAGGCGGTTCGTAGAGCTGCTGT
TCTAGAATATTTGACAGCTGAAATTCTTGAGTTGGCTAGTACTATCGAGGAGCAAGCC
AAAACATTTCTTGATAAGTTTAACCACGAGGCTGAGGACCTTTTTTATCAATCCTCTTT
GGCCTCCTGGAATTATAATACTAACATCACCGAGGAGAATGTTCAAAATATGAACAAC
GCTGGGGATAAGTGGTCTGCATTTCTCAAGGAGCAATCTACACTTGCACAGATGTACC
CACTGCAGGAA.

(2) DNA sequence of GLP:

(SEQ ID NO: 47)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTCATG
TAGTTTGGCACCGCGAATCTCCGTCCGGTCAGACCGATACCCTGGCTGCATTTCCGGA
AGACCGCTCTCAGCCGGGTCAGGATGCTCGTTTCCGCGTTACCCAGCTGCCTAACGG
CCGTGATTTCCACATGAGCGTCGTGCGTGCCCGCCGTAACGATTCCGGTACCTACGTA
TGCGGCGTTATTTCTCTGGCGCCGAAAATCCAGATCAAAGAAAGC.

(3) DNA sequence of GLPH:

(SEQ ID NO: 48)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTGGTT
TTACTTTCAGCAGCTACATTATGATGTGGGTACGTCAGGCACCGGGTAAAGGCCTGGA
GTGGGTAAGCAGCATCTATCCGAGCGGTGGTATCACCTTCTACGCTGACACCGTCAAA
GGTCGTTTCACCATCTCTCGCGACAACAGCAAGAACACCCTGTACCTGCAGATGAAC
TCCCTGCGTGCGGAGGATACTGCAGTCTACTATTGCGCCCGTATCAAACTGGGTACGG
TCACTACC.

(4) DNA sequence of GLPL:

(SEQ ID NO: 49)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTAGCG
ATTCTTGGATTCACTGGGTACGTCAGGCTCCGGGCAAAGGTCTGGAATGGGTTGCATG
GATTTCTCCGTACGGCGGTTCCACGTACTATGCTGACTCCGTGAAAGGTCGCTTCACG
ATCTCCGCAGACACCTCCAAAAACACCGCCTACCTGCAGATGAATTCTCTGCGCGCG
GAAGACACCGCAGTATACTACTGCGCTCGTCGCCACTGG.

(5) DNA sequence of GLAm:

(SEQ ID NO: 50)
ATGGTTGAGCCGCAGAAATTCGCCGAAGAACTGATCCACCGTCTGGAAG
CTGTTCAGCGTGCGGCCGTGCTGGAATACCTGACTGCAGAAATCCTGGA
ACTGGCTGCGATGTCTTGGGTTCGCCAAGCACCAGGCAAAGGCCTGGAA
TGGGTGAGCGCGATCAACGCTTCTGGTACCCGCACCTACTACGCGGACT
CCGTTAAA.

(6) DNA sequence of GLAmH:

(SEQ ID NO: 51)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTGGTT
TCACTTTCTCCCGCTACTCTATGTCCTGGGTTCGTCAGGCTCCGGGTAAAGGCCTGGA
ACTGGTTGCGCAGATCAACTCCGTTGGTAGCTCTACTTACTATCCGGACACGGTTAAG
GGTCGTTTCACCATCTCCCGTGATAACGCGAAAAACACGCTGTACCTGCAGATGAATT
CTCTGCGTGCTGAAGATACTGCGGTTTACTATTGTGCATCCGGTGACTAC.

(7) DNA sequence of GLAmM:

(SEQ ID NO: 52)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTTGGA
TCGAATGGATTAAACAGCGCCCAGGTCATGGTCTGGAATGGATCGGTGAAGTACTGC
CGGGTTCTGGTAAGTCTAACCACAACGCGAACTTCAAAGGCCGTGCCACTTTCACTG
CAGATACTGCTTCCAACACGGCGTATATGCAGCTGTCTTCCCTGACCTCCGAAGATTC
TGCGGTTTACTACTGCGCTCGTGAAGGCTCTAACAACAAC.

(8) DNA sequence of GLC:

(SEQ ID NO: 53)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTAGTA
CTATCGAGGAGCAAGCCAAAACATTTCTTGATAAGTTTAACCACGAGGCTGAGGACC
TTTTTTATCAATCCTCTTTGGCCTCCTGGAATTATAATACTAACATCACCGAGGAGAAT
GTTCAAAATATGAACAACGCTGGGGATAAGTGGTCTGCATTTCTCAAGGAGCAATCTA
CACTTGCACAGATGTACCCACTGCAGGAA.

(9) DNA sequence of GLCH:

(SEQ ID NO: 54)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTGGTC
GTTCTCTGCGTCTGAGCTGTGCCGCATCTGGTTTCACTTTTGATGATTACGCTATGCAT
TGGGTGCGTCAAGCGCCAGGTAAAGGTCTGGAATGGGTTTCTGGCGTGAGCTGGAAC
TCTGGTACCATCGGCTATGCGGATTCCGTAAAGGGTCGTTTCACTATCAGCCGCGATA
ACGCGAAGAACTCCCTGTACCTGCACATGCGTTCCCTGCGTGCAGAAGACACCGCGC
TGTACTACTGCGCCAAAGCAGTAGAAATGGTTCGCGGCCTGATGGGTCTGGGTGCAG
ATCCGGAA.

(10) DNA sequence of GLCM:

(SEQ ID NO: 55)
ATGGTTGAGCCGCAGAAATTCGCCGAAGAACTGATCCACCGTCTGGAAGCTG
TTCAGCGTGCGGCCGTGCTGGAATACCTGACTGCAGAAATCCTGGAACTGGCTTCCT
CTGCAGTAGCATGGTACCAACAGAAACCGGGTAAAGCGCCGAAACTGCTGATTTACT
CTGCGTCTGATCTGTACTCCGGCGTTCCTTCCCGTTTTAGCGGTTCTCGTTCTGGCACC
GACTTCACCCTGACCATCTCTTCTCTGCAGCCAGAAGACTTCGCGACTTACTACTGCC
AGCAATCCTACCGT.

### 5. Gene Cloning of all GPTACs

### (1) Primer Design

Primers with restriction enzyme sites at both ends were designed according to the sequence characteristics and ligated to a pET41b vector to generate a C-terminal His tag. The upstream primer had an NdeI restriction site, and the downstream primer had a HindIII restriction site. All primers were entrusted to Sangon Biotech (Shanghai) Co., Ltd. for synthesis.
(a). Primer design for BLC expression vector (pET41b-BLC-His):

upstream primer:
(SEQ ID NO: 56)
AGGAGATATACATATGGAGGAATTAATAGATAGAAGTC;
and
downstream primer:
(SEQ ID NO: 57)
GTGCGGCCGCAAGCTTTTCCTGCAGTGGGTACATC.

(b). Primer design for GLP expression vector (pET41b-GLP-His):

upstream primer:
(SEQ ID NO: 58)
AGGAGATATACATATGGTAGAACCGCAGAAATTCG;
and
downstream primer:
(SEQ ID NO: 59)
GTGCGGCCGCAAGCTTGCTTTCTTTGATCTGGATTTT.

(c). Primer design for GLPH expression vector (pET41b-GLPH-His):

upstream primer:
(SEQ ID NO. 60)
ATCCTGGAACTGGCTGGTTTTACTTTCAGCAGCTA;
and
downstream primer:
(SEQ ID NO. 61)
TGCGGCCGCAAGCTTGGTAGTGACCGTACCCAG.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(d). Primer design for GLPL expression vector (pET41b-GLPL-His):

upstream primer:
(SEQ ID NO. 64)
ATCCTGGAACTGGCTAGCGATTCTTGGATTCACTG;
and
downstream primer:
(SEQ ID NO. 65)
TGCGGCCGCAAGCTTCCAGTGGCGACGAGCG.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(e). Primer design for GLAm expression vector (pET41b-GLAm-His):

### Upstream Primer:

(SEQ ID NO: 66)
ATCCTGGAACTGGCTGCGATGTCTTGGGTTCGCCAAGCACCAGGCAAAG
GCCTGGAATGGGTGAGCGCGAT;
downstream primer:
(SEQ ID NO: 67)
TGCGGCCGCAAGCTTTTTAACGGAGTCCGCGTAGTAGGTGCGGGTACCA
GAAGCGTTGATCGCGCTCACCCAT
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(f). Primer design for GLAmH expression vector (pET41b-GLAmH-His):

upstream primer:
(SEQ ID NO. 68)
ATCCTGGAACTGGCTGGTTTCACTTTCTCCCGCT;
and
downstream primer:
(SEQ ID NO. 69)
TGCGGCCGCAAGCTTGTAGTCACCGGATGCACAA.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(g.). Primer design for GLAmM expression vector (pET41b-GLAmM-His):

upstream primer:
(SEQ ID NO. 70)
ATCCTGGAACTGGCTTGGATCGAATGGATTAAACAG;
and
downstream primer:
(SEQ ID NO. 71)
TGCGGCCGCAAGCTTGTTGTTGTTAGAGCCTTCAC.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(h). Primer design for GLC expression vector (pET41b-GLC-His):

upstream primer:
(SEQ ID NO: 72)
AGGAGATATACATATGGTTGAACCGCAGAAATTCG;
and
downstream primer:
(SEQ ID NO: 73)
GTGCGGCCGCAAGCTTTTCCTGCAGCGGGTACATT.

(c). Primer design for GLCH expression vector (pET41b-GLCH-His):

upstream primer:
(SEQ ID NO. 74)
ATCCTGGAACTGGCTGGTCGTTCTCTGCGTCTG;
and
downstream primer:
(SEQ ID NO. 75)
TGCGGCCGCAAGCTTTTCCGGATCTGCACCCAG.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

(j). Primer design for GLCM expression vector (pET41b-GLCM-His):

upstream primer:
(SEQ ID NO. 76)
ATCCTGGAACTGGCTTCCTCTGCAGTAGCATGGT;
and
downstream primer:
(SEQ ID NO. 77)
TGCGGCCGCAAGCTTACGGTAGGATTGCTGGCA.
pET41b-GL-His backbone upstream primer:
(SEQ ID NO: 62)
AAGCTTGCGGCCGCAC;
and
pET41b-GL-His backbone downstream primer:
(SEQ ID NO: 63)
AGCCAGTTCCAGGATTTCT.

### (2) PCR Amplification of the Target Gene

In a 50 μL PrimeSTAR Max (TAKARA, the preparation method may be referred to reagent instructions) reaction system, all upstream and downstream primer pairs of GPTAC used the synthesized CDS as a template. Some GPTACs contained pET41b-GL-His backbone upstream and downstream primer pairs, which were used to amplify the corresponding backbone sequence using the vector pET41b-GLP-His as a template. The primers were subjected to PCR amplification, and the PCR reaction conditions were: initial denaturation at 98° C. for 4 min; thermal cycle at 98° C. for 30 s, annealing at 55° C. for 15 s, extension at 72° C. for 5 s/kb, 30 cycles; and extension at 72° C. for 10 min.

(3) The target fragment was amplified by PCR. After confirming the DNA amplification product of the target gene by 1% agarose gel electrophoresis, an obtained amplified product was recovered and purified using a PCR product purification kit (DR02-PCR, Aidlab).

### 6. Construction of a Recombinant Plasmid

A construction method was the same as that in Example 1.

The constructed recombinant plasmids pET41b-BLC-His, pET41b-GLP-His, pET41b-GLPH-His, pET41b-GLPL-His, pET41b-GLAm-His, pET41b-GLAmH-His, pET41b-GLAmM-His, pET41b-GLC-His, pET41b-GLCH-His, and pET41b-GLCM-His were entrusted to Beijing Tsingke Biotech Co., Ltd. for sequencing and identification. The detected sequences were compared with the corresponding references and were completely matched. This indicated that the recombinant plasmid was constructed successfully.

### 7. Expression of Recombinant Plasmid for all GPTACs in Host Bacteria

A construction method was the same as that in Example 1.

### 8. Purification of Recombinant Protein

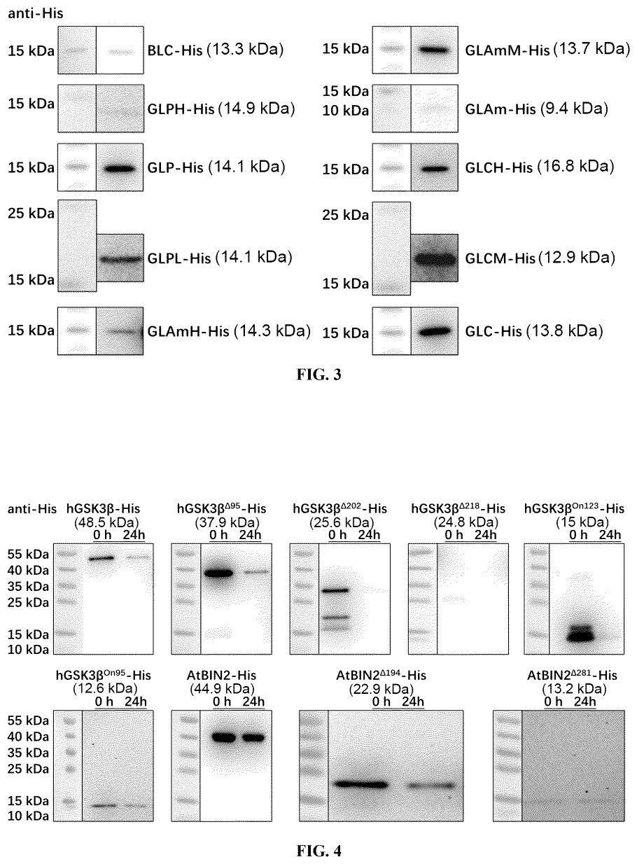

A construction method was the same as that in Example 1. The proteins produced by the recombinant plasmid pET41b-BLC-His, pET41b-GLP-His, pET41b-GLPH-His, pET41b-GLPL-His, pET41b-GLAm-His, pET41b-GLAmH-His, pET41b-GLAmM-His, pET41b-GLC-His, pET41b-GLCH-His, and pET41b-GLCM-His were named BLC-His, GLP-His, GLPH-His, GLPL-His, GLAm-His, GLAmH-His, GLAmM-His, GLC-His, GLCH-His, and GLCM-His, respectively. As shown in FIG. 3, all recombinant proteins were purified correctly.

### Example 4

The hGSK3β, domain-knockout hGSK3β variants, single domain hGSK3β variants, AtBIN2, and single domain AtBIN2 variants showed protease activity.
1. A concentration of the recombinant protein was measured using a Nanodrop micro-spectrophotometer.
2. Conversion of molar concentration of recombinant protein: After logging in to the website web.expasy.org/protparam/, the amino acid sequence of the recombinant protein was entered to check the absorbance value Abs. For each recombinant protein, it was defined that the molar concentration was M (mM/L), the substance concentration was C (mg/mL), the light absorption value was A (0.1%=1 g/L), and the molecular weight was M (kD). A conversion formula for the molar concentration of the recombinant protein was: M=(C/A)*1000/M or M=1000C/(A*M).
3. Detection scheme of the protease activity:

The recombinant protein obtained in Example 1 was tested for protease activity. A specific process was as follows:

### Reaction System (22 μL):

(1) Dialysis Buffer: 20 mM Tris HCl (pH=7.5), 20 mM NaCl, 10 mM MgCl₂, and 0.5 mM KCl.
(2) ATP: final concentration: 1 mM.
(3) hGSK3β, AtBIN and variant proteins: final concentration 20 μM, dialyzed against Dialysis buffer.

### Sample Injection Order:

(1) Dialysis Buffer. (2) ATP. (3) hGSK3β or AtBIN or variant proteins.

### Precautions:

(1) The reaction system required an ice bath. (2) Each reagent should be mixed immediately after addition. (3) For each treatment, the EP tubes “0 h” and “24 h” were marked and 10 μL of 2×SDS protein loading buffer was added to the “0 h” EP tube in advance. (4) After the reaction system was prepared, 10 μL of the reaction sample was dispensed into the “0 h” and “24 h” EP tubes and mixed quickly.

### Reaction Conditions:

(1) Human GSK3β (hGSK3β) and its variants were incubated at 37° C. for 24 h.
(2) Arabidopsis thaliana BIN2 (AtBIN2) and its variants were incubated at 28° C. for 24 h.
(3) After the incubation, 10 μL of 2×SDS protein loading buffer was added to the “24 h” EP tube and mixed quickly.

### Detection Conditions:

(1) Each treated sample pair (“0 h” and “24 h”) was sequentially loaded onto 15% SDS-PAGE (Tris-Glycine system), and the stacking gel was run at 220 V for 6 min, and the separation gel was run at 280 V for 15 min.
(2) Conventional semi-dry transfer procedure: 2×0.22 μm nitrocellulose membrane, 0.8×membrane area (mm²) mA current, run for 30 min to 40 min (requiring determination by preliminary experiments).
(3) The membrane was immersed in PBST-M (5% milk, 0.1% Tween-20 in 1×PBS) and blocked for 1 h.
(4) HRP-conjugated anti-His-tagged mouse monoclonal antibody was added (Beyotime, AF2873-200 μL, 1:5000 dilution) and incubated for 1 h.
(5) PBST-M was discarded, the product was quickly washed 3 times with PBST (0.1% Tween-20 in 1×PBS), and then washed with PBST for 10 min, 5 min, and 5 min.
(6) Exposure and development were conducted.
4. The results are shown in FIG. 4. Most of the recombinant proteins had obvious protease activity. Only AtBIN2^{Δ281}-His showed weak protease activity. It should be noted that AtBIN2^{On95}-His was not shown in FIG. 4 because its protease activity was too strong. Even in the ice bath operation, there was no AtBIN2^{On95}-His signal in all “0 h” samples.

### Example 5

Factors Affecting the Protease Activity of hGSK3β
1. In vitro target protein proteolysis test was conducted using hGSK3β as a research object:

### Reaction System (22 μL):

(1) Dialysis Buffer: 20 mM Tris HCl (pH=7.5), 20 mM NaCl, 10 mM MgCl₂, and 0.5 mM KCl.
(2) ATP: final concentration: 1 mM.
(3) Protease activity treatment agent was: MG132 (S1748-5 mg, final concentration 100 μM, Beyotime) or Bikinin (HY-12524-5 mg, final concentration 30 μM, MedChemExpress) or Cisplatin (S1552, final concentration 150 μM, Beyotime) or AEBSF (SG2000-5 mg, final concentration 1 mM, Beyotime) or Pepstain A (SG2016-5 mg, final concentration 1 mM, Beyotime) or Leupeptin (L8110, final concentration 1 mM, Solarbio) or 1,10-Phenanthrolin (Δ600693-0005, final concentration 1 mM, Sangon Biotech (Shanghai) Co., Ltd.).
(4) hGSK3β and its variant proteins: final concentration 20 μM, dialyzed against Dialysis buffer.

### Sample Injection Order:

(1) Dialysis Buffer. (2) ATP. (3) Protease activity treatment agent. (4) hGSK3β or its variant proteins.

### Precautions:

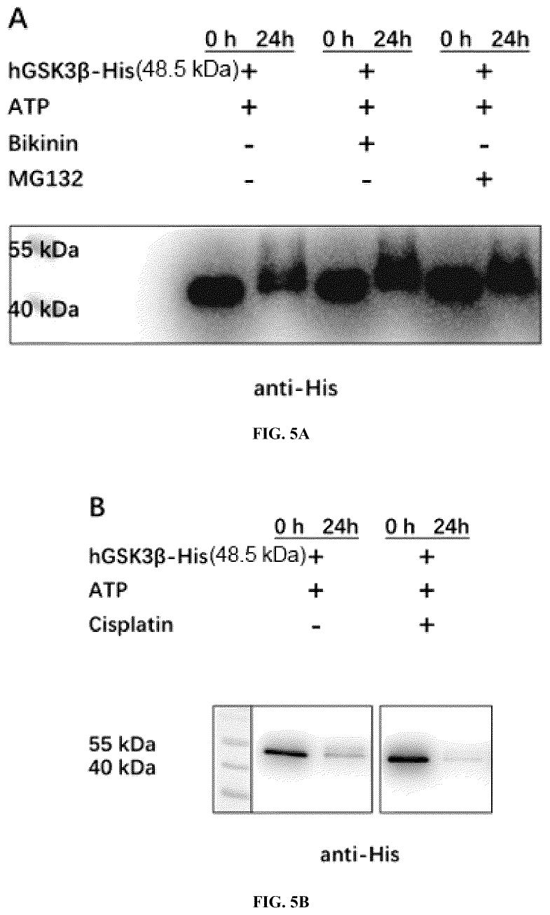

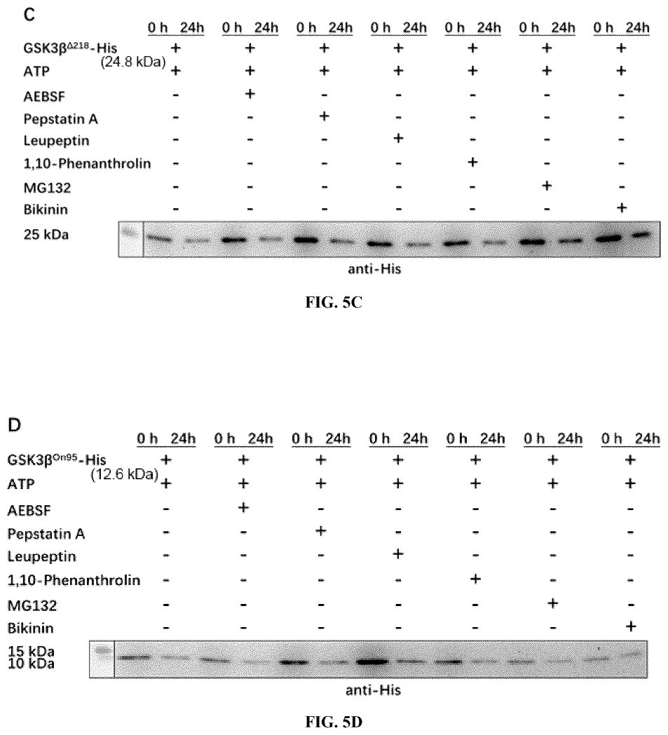

(1) The reaction system required an ice bath. (2) Each reagent should be mixed immediately after addition. (3) The treatment group settings are shown in FIG. 5.
(4) For multiple treatments, bulk samples containing Dialysis Buffer and ATP must be prepared, and the least volume of the Dialysis Buffer among all treatment groups should be used as the standard. For example, if there were 7 treatments, then the bulk samples should be prepared with (7.2 to 7.6) times the Dialysis Buffer and ATP in the treatment group containing the least Dialysis Buffer, then mixed and aliquoted.
(5) For each treatment group, it was compensated for the missing Dialysis Buffer and then added with the corresponding protease activity treatment agent to ensure that a total volume of the reaction system was always 22 μL.
(6) For each treatment, the EP tubes “0 h” and “24 h” were marked and 10 μL of 2×SDS protein loading buffer was added to the “0 h” EP tube in advance.
(7) After the reaction system was prepared, 10 μL of the reaction sample was dispensed into the “0 h” and “24 h” EP tubes and mixed quickly.

### Reaction Conditions:

(1) Human GSK3β (hGSK3β) and its variants were incubated at 37° C. for 24 h.
(2) After the incubation, 10 μL of 2×SDS protein loading buffer was added to the “24 h” EP tube and mixed quickly.

### Detection Conditions:

(1) Each treated sample pair (“0 h” and “24 h”) was sequentially loaded onto 15% SDS-PAGE (Tris-Glycine system), and the stacking gel was run at 220 V for 6 min, and the separation gel was run at 280 V for 15 min.
(2) Conventional semi-dry transfer procedure: 2×0.22 μm nitrocellulose membrane, 0.8×membrane area (mm²) mA current, and run for 30 min to 40 min (requiring determination by preliminary experiments).
(3) The membrane was immersed in PBST-M (5% milk, 0.1% Tween-20 in 1×PBS) and blocked for 1 h.
(4) HRP-conjugated anti-His-tagged mouse monoclonal antibody was added (Beyotime, AF2873-200 μL, 1:5000 dilution) and incubated for 1 h.
(5) PBST-M was discarded, the product was quickly washed 3 times with PBST (0.1% Tween-20 in 1×PBS), and then washed with PBST for 10 min, 5 min, and 5 min.
(6) Exposure and development were conducted.
2. MG132, Bikinin, and Cisplatin were added into the reaction system as control groups. MG132 was dissolved in DMSO to a final concentration of 100 μM. Bikinin was dissolved in DMSO to a final concentration of 30 μM. Cisplatin was dissolved in 1×PBS to a final concentration of 150 μM.
3. As shown in FIG. 5A, MG132 and Bikinin could strongly inhibit the protease activity of hGSK3β-His, and both MG132 and Bikinin could inhibit three independent domains of hGSK3β (hGSK3β^{Δ218}-His, hGSK3β^{On95}-His, hGSK3β^{On123}-His) (FIG. 5C, FIG. 5D, FIG. 5E). As shown in FIG. 5B, Cisplatin could significantly enhance the protease activity of hGSK3β-His. When analyzing the protease properties of each domain of hGSK3, AEBSF (serine protease inhibitor), Pepstatin A (aspartate protease inhibitor), Leupeptin (serine protease, threonine protease, and cysteine protease inhibitor), 1,10-Phenanthrolin (metalloprotease (carboxypeptidase) inhibitor), MG132 (proteasome inhibitor, which could inhibit the activity of serine protease, calpain (calcium-dependent cysteine endopeptidase), etc.) were used. In addition, Bikinin (a plant GSK3 kinase activity inhibitor) was also used. As shown in FIG. 5C, the N-terminal domain of hGSK3 had a strong protease activity. Compared to the active inhibitor, the domain had already self-degraded by more than half in just a few seconds of preparation time. All 6 inhibitors had strong inhibitory effects on this domain. Therefore, this domain must be serine, aspartate, and metalloprotease, and might contain threonine and cysteine protease activities, and was inhibited by MG132 and Bikinin. As shown in FIG. 5D, the intermediate domain of hGSK3 had a strong protease activity. Compared to the active inhibitor, the domain had already self-degraded by more than half in just a few seconds of preparation time. Pepstatin A had a certain inhibitory effect, while Leupeptin had a highly strong inhibitory effect. Therefore, this domain must be aspartate protease and must contain one or more of serine, threonine, and cysteine protease activities. As shown in FIG. 5E, the protease activity of the C-terminal domain of hGSK3 was extremely weak, but its 24-h treatment also reflected the effect of the corresponding inhibitor. AEBSF, Pepstatin A, and Bikinin all showed a certain inhibitory effect. Although MG132 also had a certain inhibitory effect, its instantaneous inhibitory effect was less than that of the first three. Therefore, this domain must be serine and aspartate protease, might contain cysteine protease activity, and was inhibited by MG132 and Bikinin.

### Example 6

### GPTAC Guiding GSK3 to Efficiently Degrade Target Proteins

1. BLC-His guided AtBIN2-His to degrade novel coronavirus spike glycoprotein (COVID-19 Spike RBD).
1) AtBIN2-His was used as a protease. 2) The BLC-His purified in Example 3 was used. 3) A commercial novel coronavirus spike glycoprotein (Sino Biological, 40592-V05H) was used, named NS-mFc. 4) In vitro target protein proteolysis scheme: AtBIN2-His, BLC-His, and NS-mFc were used as reaction reagents, and the proteolysis of NS-mFc was detected.

GPTAC-guided GSK3 proteolysis scheme of target protein (Example 6, all shared this experimental scheme):

### Reaction System (22 μL):

(1) Dialysis Buffer: 20 mM Tris HCl (pH=7.5), 20 mM NaCl, 10 mM MgCl₂, and 0.5 mM KCl.
(2) ATP: final concentration: 1 mM.
(3) Target protein: final concentration 20 μM, dialyzed against Dialysis buffer.
(4) AtBIN2 or hGSK3p: final concentration 20 μM, dialyzed against Dialysis buffer.
(5) GPTAC: final concentration 200 μM, dialyzed against Dialysis buffer.

### Sample Injection Order:

(1) Dialysis Buffer. (2) ATP. (3) Target protein. (4) AtBIN2 or hGSK3β.
(5) GPTAC.

### Precautions:

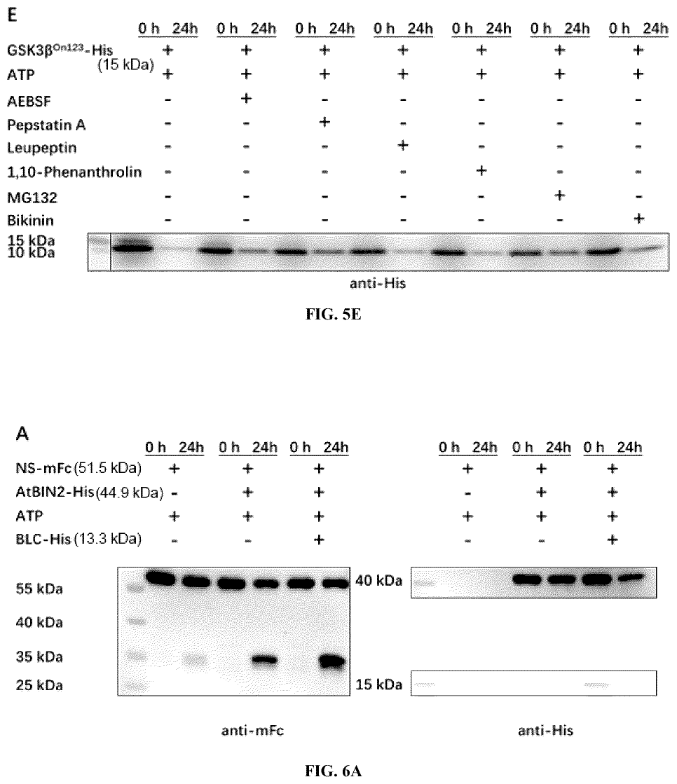

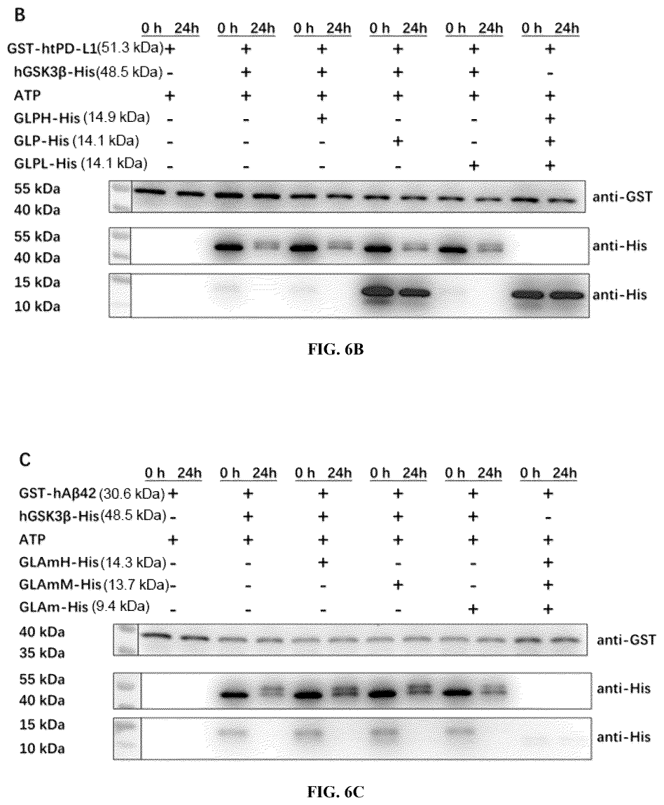

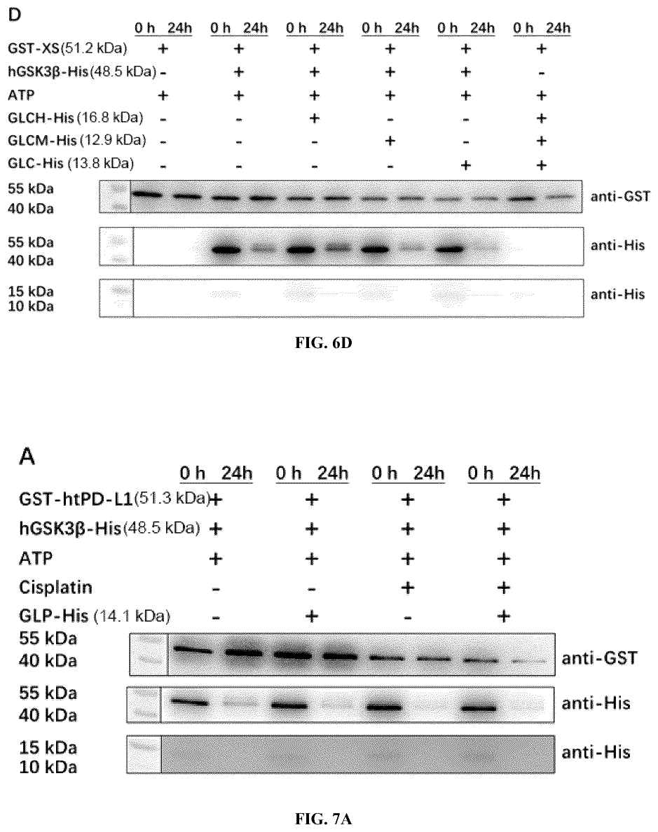

(1) The reaction system required an ice bath. (2) Each reagent should be mixed immediately after addition. (3) The treatment group settings are shown in FIG. 6. (4) For multiple treatments, bulk samples containing Dialysis Buffer, ATP, and target protein must be prepared, and the least volume of the Dialysis Buffer among all treatment groups should be used as the standard. For example, if there were 7 treatments, then the bulk samples should be prepared with (7.2 to 7.6) times the Dialysis Buffer, ATP, and target protein in the treatment group containing the least Dialysis Buffer, then mixed and aliquoted. (5) For each treatment group, losing Dialysis Buffer was supplemented to ensure that the total volume of the reaction system was always 22 μL. (6) The corresponding AtBIN2 or hGSK3β was added, mixed well and quickly, and GPTAC was added and mixed well and quickly. (7) For each treatment, the EP tubes “0 h” and “24 h” were marked and 10 μL of 2×SDS protein loading buffer was added to the “0 h” EP tube in advance. (8) After the reaction system was prepared, 10 μL of the reaction sample was dispensed into the “0 h” and “24 h” EP tubes and mixed quickly.

### Reaction Conditions:

(1) System containing human GSK3β (hGSK3β) was incubated at 37° C. for 24 h.
(2) System containing Arabidopsis thaliana BIN2 (AtBIN2) was incubated at 28° C. for 24 h.
(3) After the incubation, 10 μL of 2×SDS protein loading buffer was added to the “24 h” EP tube and mixed quickly.

### Detection Conditions:

(1) Each treated sample pair (“0 h” and “24 h”) was sequentially loaded onto 15% SDS-PAGE (Tris-Glycine system), and the stacking gel was run at 220 V for 6 min, and the separation gel was run at 280 V for 15 min.
(2) Conventional semi-dry transfer procedure: 2×0.22 μm nitrocellulose membrane, 0.8×membrane area (mm²) mA current, run for 30 min to 40 min (requiring determination by preliminary experiments).
(3) The membrane was immersed in PBST-M (5% milk, 0.1% Tween-20 in 1×PBS) and blocked for 1 h.
(4) HRP-conjugated anti-GST-tagged mouse monoclonal antibody was added (Beyotime, AF2891-50 μL, 1:5000 dilution) and incubated for 1 h.
(5) PBST-M was discarded, the product was quickly washed 3 times with PBST (0.1% Tween-20 in 1×PBS), and then washed with PBST for 10 min, 5 min, and 5 min.
(6) Exposure and development were conducted.
(7) After development, the membrane was washed quickly 3 times with PBST (0.1% Tween-20 in 1×PBS), and then washed with PBST for 30 min to remove most of the HRP-conjugated anti-GST-tagged mouse monoclonal antibody (the amount could be adjusted appropriately depending on the clearing situation).
(8) The membrane was immersed in PBST-M (5% milk, 0.1% Tween-20 in 1×PBS) and blocked for 1 h.
(9) HRP-conjugated anti-His-tagged mouse monoclonal antibody was added (Beyotime, AF2873-200 μL, 1:5000 dilution) and incubated for 1 h.
(10) PBST-M was discarded, the product was quickly washed 3 times with PBST (0.1% Tween-20 in 1×PBS), and then washed with PBST for 10 min, 5 min, and 5 min.
(11) Exposure and development were conducted.
5) As shown in FIG. 6A, compared with the control, BLC-His could guide AtBIN2-His to cleave more NS-mFc, thereby generating more protein fragments (approximately 35 kDa). In conclusion, BLC-His could guide AtBIN2-His to effectively degrade NS-mFc.
2. GLP-His, GLPH-His, and GLPL-His guiding hGSK3β-His to degrade GST-htPD-L1
(1) hGSK3β-His was used as a protease.
(2) GLP-His, GLPH-His, and GLPL-His purified in Example 3 were used.
(3) GST-htPD-L1 purified in Example 2 was used.
(4) The in vitro target protein proteolysis scheme was the same as above (Example 6, Part 1, Step 4)), hGSK3β-His, GLP-His or GLPH-His or GLPL-His, GST-htPD-L1 were used as reaction reagents, and the proteolysis of GST-htPD-L1 was detected.
(5) As shown in FIG. 6B, compared with the control 0 h treatment, GLP-His, GLPH-His, and GLPL-His completed the proteolysis of GST-htPD-L1 the moment they were added to the reaction system (there was almost no difference between the 0 h and 24 h treatments, indicating that the impurities contained in the protein expressed and purified by E. coli had no proteolysis effect on the target protein). In conclusion, GLP-His, GLPH-His, and GLPL-His could guide hGSK3β-His to efficiently degrade GST-htPD-L1.

Conclusion: based on the existing GPTAC targeting htPD-L1, the difference in affinity between GPTAC and htPD-L1 did not affect the proteolysis of htPD-L1 by hGSK3β.

For polypeptide-type GPTAC, the ligand and linker of GSK3 were fixed, and it was only necessary to replace the ligand of the target protein to design a GPTAC for any target protein. Since the change in affinity between the target protein (htPD-L1) ligand and the target protein (htPD-L1) did not affect the proteolysis efficiency of GSK3 on the target protein (htPD-L1), the design threshold of the target protein ligand could be relatively low. To some extent, any interacting protein may be sufficient. If there was no crystal structure of the interaction between two interacting proteins, the contact surface could be predicted using software such as Alphafold3, and then a structural domain could be selected to design the GPTAC targeting a specific target protein. Therefore, this conclusion proved the simplicity of the system of the present disclosure in terms of design and application.
3. GLAm-His, GLAmH-His, and GLAmM-His guiding hGSK3β-His to degrade GST-hAβ42
(1) hGSK3β-His was used as a protease.
(2) GLAm-His, GLAmH-His, and GLAmM-His purified in Example 3 were used.
(3) GST-hAβ42 purified in Example 2 was used.
(4) The target protein proteolysis scheme in vitro was the same as above (Example 6, Part 1, Step 4)), hGSK3β-His, GLAm-His or GLAmH-His or GLAmM-His, GST-hAβ42 were used as reaction reagents, and the proteolysis of GST-hAβ42 was detected.
(5) As shown in FIG. 6C, compared with the control, GLAm-His, GLAmHv, and GLAmM completed the proteolysis of GST-hAβ42 the moment they were added to the reaction system (there was almost no difference between 0 h and 24 h treatments). In conclusion, GLAm-His, GLAmH-His, and GLAmM-His could guide hGSK3β-His to efficiently degrade GST-hAβ42. The conclusion was the same as that in Part 2.
4. GLC-His, GLCH-His, GLCM-His guiding hGSK3β-His to degrade GST-XS
(1) hGSK3β-His was used as a protease.
(2) GLC-His, GLCH-His, and GLCM-His purified in Example 3 were used.
(3) GST-XS purified in Example 2 was used.
(4) The target protein proteolysis scheme in vitro was the same as above (Example 6, Part 1, Step 4)), hGSK3β-His, GLC-His or GLCH-His or GLCM-His, GST-XS were used as reaction reagents, and the proteolysis of GST-XS was detected.
(5) As shown in FIG. 6D, compared with the control, GLC-His, GLCH-His, and GLCM-His completed the proteolysis of GST-XS the moment when they were added to the reaction system (there was almost no difference between the 0 h and 24 h treatments). In conclusion, GLC-His, GLCH-His, and GLCM-His could guide hGSK3β-His to efficiently degrade GST-XS. The conclusion is the same as that in Part 2.

### Example 7

Cisplatin promoting the proteolysis of htPD-L1, hAβ42, and XS through hGSK3β induced by GLP, GLAmM, and GLCM, respectively
1. hGSK3β-His was used as a protease.
2. GLP-His, GLAmM-His, and GLCM-His purified in Example 3 were used.
(3) GST-htPD-L1, GST-hAβ42, and GST-XS purified in Example 2 were used.
4. Three reaction combinations: “hGSK3β-His, GLP-His, GST-htPD-L1”, “hGSK3β-His, GLAmM-His, GST-hAβ42”, and “hGSK3β-His, GLCM-His, GST-XS” were set up to detect the promoting effect of Cisplatin on GPTAC-guided hGSK3β proteolysis of the corresponding target protein. Specific steps were as follows:

### Reaction System (22 μL):

(1) Dialysis Buffer: 20 mM Tris HCl (pH=7.5), 20 mM NaCl, 10 mM MgCl₂, and 0.5 mM KCl.
(2) ATP: final concentration: 1 mM.
(3) Target protein: final concentration 20 μM, dialyzed against Dialysis buffer.
(4) hGSK3β: final concentration 20 μM, dialyzed against Dialysis buffer.
(5) Cisplatin: final concentration: 150 μM.
(6) GPTAC: final concentration 200 μM, dialyzed against Dialysis buffer.

### Sample Injection Order:

(1) Dialysis Buffer. (2) ATP. (3) Target protein. (4) hGSK3β. (5) GPTAC and/or Cisplatin.

### Precautions:

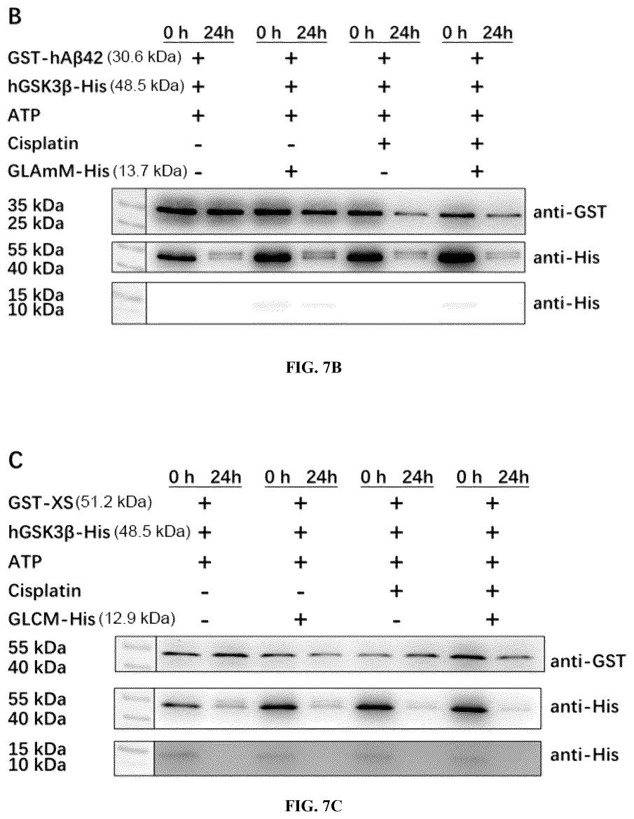

(1) The reaction system required an ice bath. (2) Each reagent should be mixed immediately after addition. (3) The treatment group settings are shown in FIG. 7. (4) For multiple treatments, bulk samples containing Dialysis Buffer, ATP, target protein, and hGSK3β must be prepared, and the least volume of the Dialysis Buffer among all treatment groups should be used as the standard. For example, if there were 7 treatments, then the bulk samples should be prepared with (7.2 to 7.6) times the Dialysis Buffer, ATP, target protein, and hGSK3β in the treatment group containing the least Dialysis Buffer, then mixed and aliquoted. (5) For each treatment group, it was compensated for the missing Dialysis Buffer to ensure that a total volume of the reaction system was always 22 μL. (6) The corresponding Cisplatin and/or GPTAC were added, mixed well and quickly. (7) For each treatment, the EP tubes “0 h” and “24 h” were marked and 10 μL of 2×SDS protein loading buffer was added to the “0 h” EP tube in advance. (8) After the reaction system was prepared, 10 μL of the reaction sample was dispensed into the “0 h” and “24 h” EP tubes and mixed quickly.

### Reaction Conditions:

(1) System containing human GSK3β (hGSK3β) was incubated at 37° C. for 24 h.
(2) After the incubation, 10 μL of 2×SDS protein loading buffer was added to the “24 h” EP tube and mixed quickly.

### Detection Conditions:

(1) Each treated sample pair (“0 h” and “24 h”) was sequentially loaded onto 15% SDS-PAGE (Tris-Glycine system), and the stacking gel was run at 220 V for 6 min, and the separation gel was run at 280 V for 15 min.
(2) Conventional semi-dry transfer procedure: 2×0.22 μm nitrocellulose membrane, 0.8×membrane area (mm²) mA current, and run for 30 min to 40 min (requiring determination by preliminary experiments).
(3) The membrane was immersed in PBST-M (5% milk, 0.1% Tween-20 in 1×PBS) and blocked for 1 h.
(4) HRP-conjugated anti-GST-tagged mouse monoclonal antibody was added (Beyotime, AF2891-50 μL, 1:5000 dilution) and incubated for 1 h.
(5) PBST-M was discarded, the product was quickly washed 3 times with PBST (0.1% Tween-20 in 1×PBS), and then washed with PBST for 10 min, 5 min, and 5 min.
(6) Exposure and development were conducted.
(7) After development, the membrane was washed quickly 3 times with PBST (0.1% Tween-20 in 1×PBS), and then washed with PBST for 30 min to remove most of the HRP-conjugated anti-GST-tagged mouse monoclonal antibody (the amount could be adjusted appropriately depending on the clearing situation).
(8) The membrane was immersed in PBST-M (5% milk, 0.1% Tween-20 in 1×PBS) and blocked for 1 h.
(9) HRP-conjugated anti-His-tagged mouse monoclonal antibody was added (Beyotime, AF2873-200 μL, 1:5000 dilution) and incubated for 1 h.
(10) PBST-M was discarded, the product was quickly washed 3 times with PBST (0.1% Tween-20 in 1×PBS), and then washed with PBST for 10 min, 5 min, and 5 min.
(11) Exposure and development were conducted.
5. As shown in FIG. 7A, Cisplatin directly promoted the GLP-guided transient proteolysis of htPD-L1 by hGSK3P. As shown in FIG. 7B, Cisplatin also directly promoted the GLAmM-guided transient proteolysis of hAβ42 by hGSK3P. As shown in FIG. 7C, although Cisplatin inhibited the GLCM-guided transient proteolysis of XS by hGSK3β, it could obviously exert effect for a long time. That is, Cisplatin could promote GPTAC-mediated GSK3 proteolysis of target proteins.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. (canceled)

2. (canceled)

3. (canceled)

4. (canceled)

5. (canceled)

6. (canceled)

7. A protease-based proteolysis-targeting chimera (PROTAC), wherein the protease-based PROTAC is formed by ligating two ligands with a linker, and the two ligands comprise a target protein-binding ligand and a protease-binding ligand.

8. The protease-based PROTAC according to claim 7, wherein the protease is GSK3, and the protease-based PROTAC is formed by ligating two ligands with a linker, and the two ligands comprise a target protein-binding ligand and a GSK3-binding ligand.

9. The protease-based PROTAC according to claim 8, wherein when the GSK3-binding ligand is a polypeptide and GSK3 is a human protein, the GSK3-binding ligand comprises the amino acid sequence shown in SEQ ID NO: 1.

10. The protease-based PROTAC according to claim 8, wherein when the GSK3-binding ligand is a polypeptide and GSK3 is an Arabidopsis thaliana protein, the GSK3-binding ligand comprises the amino acid sequence shown in SEQ ID NO: 2.

11. The protease-based PROTAC according to claim 8, wherein the linker comprises the amino acid sequence shown in SEQ ID NO: 3.

12. The protease-based PROTAC according to claim 8, wherein when the target protein-binding ligand is a polypeptide, the target protein-binding ligand comprises the amino acid sequence encoding a protein that interacts with a target protein on a binding surface.

13. The protease-based PROTAC according to claim 12, wherein C-terminal of the amino acid sequence encoding the protein that interacts with the target protein on the binding surface is further fused with the amino acid sequence shown in SEQ ID NO: 4.

14. (canceled)

15. A method for degrading a target protein based on the protease-based PROTAC according to claim 7, comprising the following steps: mixing the protease-based PROTAC with a target protein-containing sample to allow targeted proteolysis of the target protein.

16. The method according to claim 15, wherein the protease is GSK3 and the degrading is conducted at 36° C. to 37° C. when GSK3 is a human protein.

17. The method according to claim 15, wherein the degrading is conducted at 26° C. to 28° C. when GSK3 is an Arabidopsis thaliana protein.

18. The method according to claim 15, wherein cisplatin is added into a resulting mixed system.

19. (canceled)

20. (canceled)

21. A protease-based PROTAC-containing composition, comprising the protease-based PROTAC according to claim 7 and cisplatin, wherein protease is GSK3.

22. The protease-based PROTAC according to claim 9, wherein the linker comprises the amino acid sequence shown in SEQ ID NO: 3.

23. The protease-based PROTAC according to claim 10, wherein the linker comprises the amino acid sequence shown in SEQ ID NO: 3.

24. The method according to claim 15, wherein the protease is GSK3, and the protease-based PROTAC is formed by ligating two ligands with a linker, and the two ligands comprise a target protein-binding ligand and a GSK3-binding ligand.

25. The method according to claim 15, wherein when the GSK3-binding ligand is a polypeptide and GSK3 is a human protein, the GSK3-binding ligand comprises the amino acid sequence shown in SEQ ID NO: 1.

26. The method according to claim 15, wherein when the GSK3-binding ligand is a polypeptide and GSK3 is an Arabidopsis thaliana protein, the GSK3-binding ligand comprises the amino acid sequence shown in SEQ ID NO: 2.

27. The method according to claim 15, wherein the linker comprises the amino acid sequence shown in SEQ ID NO: 3.
